(19) United States
(12) Patent Application Publication
(10) Pub. No.: US 2025/0248920 A1
(43) Pub. Date: Aug. 7, 2025

(54) LOW PH SOLID-DISSOLVABLE FIBROUS COMPOSITION

(71) Applicant: The Procter & Gamble Company, Cincinnati, OH (US)

(72) Inventors: Matthew Lawrence LYNCH, Mariemont, OH (US); Mackenzie Jean CHILDS, Cincinnati, OH (US); Kristin Rhedrick WILLIAMS, West Chester, OH (US); Brandi Nicole TRANTER, Mt. Lookout, OH (US); Brandon Philip ILLIE, Felicity, OH (US); Vighter IBERI, Mason, OH (US)

(21) Appl. No.: 19/045,634

(22) Filed: Feb. 5, 2025

Related U.S. Application Data
(60) Provisional application No. 63/550,133, filed on Feb. 6, 2024.

(51) Int. Cl. A61K 8/92; A61Q 5/00; A61Q 19/00; C11D 3/20

(52) U.S. Cl. CPC A61K 8/92; A61Q 5/00; A61Q 19/00; C11D 3/2079; C11D 2111/12; C11D 2111/24

(57) ABSTRACT

A solid-dissolvable composition comprising: crystallizing agent; water; from 10 wt. % to about 55 wt. % of the solid-dissolvable composition of an acid converting agent; and optional benefit agent; wherein the crystallizing agent is the sodium salt of saturated fatty acids having from 8 to about 12 methylene groups; wherein the crystallizing agent is the form of fibers as determined by the FIBER TEST METHOD; and wherein the water is less than about 15 wt. % of the solid-dissolvable composition.

### FIELD

A dry solid-dissolvable composition comprising a sodium fatty acid carboxylate in the form of an interlocking fiber mesh, at least one solid acid converting agent, and an optional benefit agent, which when added to water result in a low pH aqueous treatment liquor.

### BACKGROUND

A dry solid-dissolvable, single-measure composition added to water creating a low pH aqueous treatment liquor has significant utility. In one non-limiting example, the aqueous treatment liquor can be used to treat sensitive skin, where high pH treatment liquor can result in skin irritation; in another non-limiting example, the aqueous treatment liquor can be used to treat fabrics to reduce malodor resulting from bacterial growth (i.e., acid treatment); in another non-limiting example, the aqueous treatment liquor can be used on hair to providing detangling benefits. Adding the composition as a single-measure form ensures the consumer adds the proper dosage of ingredients to the treatment liquor to do the required job. In one non-limiting example, the benefit agents are at the proper levels to treat sensitive skin; in another non-limiting example, the benefit agents are added to a wash drum at the proper levels to treat fabrics. A low pH aqueous treatment liquor can deliver a compact, single-measured composition that is needed and is ideal for consumers.

A dry solid-dissolvable single-measure composition comprising a sodium fatty acid carboxylate is known in the context of refreshing fabrics (e.g., U.S. patent application Ser. No. 18/366,730). The sodium fatty acid carboxylate in such compositions form crystalline fibers (FIG. 1) during processing, which are interwoven creating a mesh microstructure (FIG. 2) that is critical to forming these viable, porous solids. Surprisingly, such crystalline fibers are shown to be both thermally stable (FIG. 3) and to be relatively insensitive to humidity (FIG. 4) in comparison to other fiber-like compositions, both critical to supply-chain and in-use stability. Further, the crystallizing agent is “bio-based” and “renewable material” enhancing the sustainability of the compositions. The porous microstructures from the mesh can hydrate and/or dissolve faster than completely solid microstructure, because of the ability to take in water. However, when added to water, the sodium fatty acid carboxylate fibers create a high pH aqueous treatment liquor (typically 9<pH<11) as they are weak bases, and which is antithetical to many of the previously described applications.

The obvious approach to solve this problem for one skilled in the art, is to covert some (or all) the sodium fatty acid carboxylate into a fatty acid (which is a weak acid) such that when the composition is added to water, a low pH aqueous treatment liquor is obtained. A composition comprising only sodium fatty acid carboxylate, when added to water, results in a wash liquor of pH=9.5 and the crystals in the dry composition are long fibers (FIG. 1). Herein, the terms “treatment liquor” and “wash liquor” are used interchangeably. In one example, a composition is prepared by replacing half of the sodium fatty acid carboxylate with fatty acid, which-when added to water, results in a wash liquor of pH=7.03 (lower pH) and the crystals in the dry composition are no longer fibers (FIG. 5). Said composition is unsuitable for this invention on the basis of high pH wash liquors and non-fiber crystals. In another example, a composition is prepared by replacing three-quarters of the sodium fatty acid carboxylate with fatty acid, which-when added to water, results in a wash liquor of pH=6.65 (even lower pH) and the crystals in the dry composition are still no longer fibers (FIG. 6). Said composition is unsuitable for this invention in that the one creates a low pH wash liquor, but on the basis of non-fiber crystals leading to soft compositions. These observations suggest that there is no way to create a composition based on sodium fatty acid carboxylate that can be added to water to create a low pH treatment liquor while maintaining crystalline fibers.

Surprisingly, the present inventors have discovered that a solid-dissolvable composition can be prepared: 1) by preparing a substrate from dried fiber crystalline mesh with sodium fatty acid carboxylate, and 2) by adding a solid acid converting agent at the right proportions as articulated in this disclosure (FIG. 7), and 3) adding an optional benefit agent, resulting in a low pH aqueous treatment liquor when placed with water while maintaining the mesh microstructure needed for the required hydrating and physical properties in convenient single-measure compositions.

### SUMMARY

A solid-dissolvable composition comprising:
crystallizing agent;
water;
from 10 wt. % to about 55 wt. % of the solid-dissolvable composition of an acid converting agent;
and optional benefit agent;
wherein the crystallizing agent is the sodium salt of saturated fatty acids having from 8 to about 12 methylene groups; and
wherein the crystallizing agent is the form of fibers as determined by the FIBER TEST METHOD;
and wherein the water is less than about 15 wt. % of the solid-dissolvable composition.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of elements in any of the exemplary embodiments, except as may be explicitly delineated in the corresponding written description. None of the drawings are necessarily to scale.

FIG. 1 is an electron micrograph of the crystalline fibers of sodium fatty acid carboxylate disclosed in U.S. patent application Ser. No. 18/366,730.

FIG. 2 is an electron micrograph of the crystalline mesh microstructure disclosed in U.S. patent application Ser. No. 18/366,730.

FIG. 3 is a graph showing the thermostability of the solid-dissolvable composition disclosed in U.S. patent application Ser. No. 18/366,730 (solid line), in comparison to a comparative example of fiber unit-dose technology (e.g., disclosed in US 2020/0299888 A1, and US 2020/0297645 A1) (dashed line), as measured by the THERMAL STABILITY TEST METHOD. The thermostability stability temperature is preferably in excess of 40° C. and more preferably in excess of preferably 60° C., and is required for supply-chain and in-use stability.

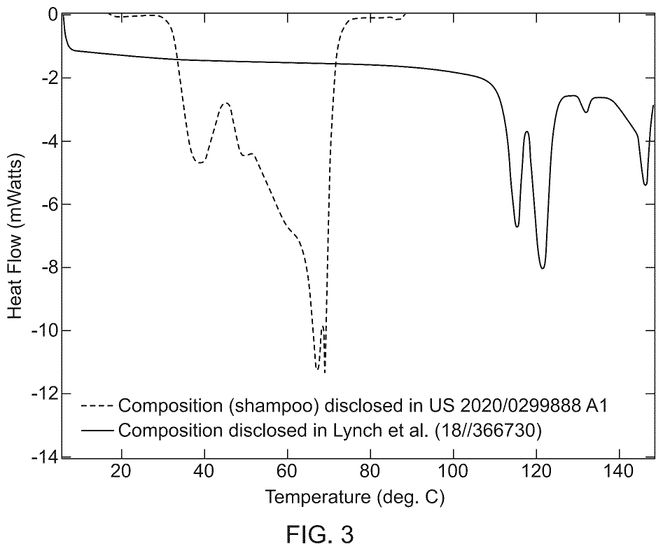

FIG. 4 is a graph showing the humidity stability of the solid-dissolvable composition disclosed in U.S. patent application Ser. No. 18/366,730, in comparison to a comparative example of fiber unit-dose technology (e.g., disclosed in US 2020/0299888 A1, and US 2020/0297645 A1), as measured by the HUMIDITY TEST METHOD. The humidity stability is preferably in excess of 70% RH and is more preferably in excess of preferably 80% RH and is required for supply-chain and in-use stability.

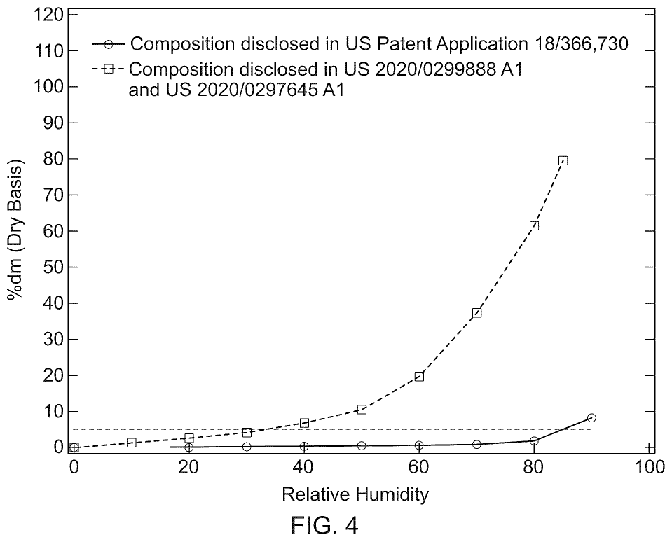

FIG. 5 is an electron micrograph of a comparative example of crystallizing agent crystals with 50 wt. % sodium and 50 wt. % hydrogen of the fatty acids resulting from under neutralization of fatty acids, in an attempt to create low pH aqueous treatment liquor. This composition results in treatment liquor with pH=7.03, as determined by the PH TEST METHOD FOR WASH LIQUOR. The crystals are no longer fibers, and the composition is no longer solid.

FIG. 6 is an electron micrograph of a comparative example of crystallizing agent crystals with 25 wt. % sodium and 75 wt. % hydrogen of the fatty acids resulting from under neutralization of fatty acids, in an attempt to create low pH aqueous treatment liquor. This composition results in treatment liquor with pH=6.65, as determined by the PH TEST METHOD FOR WASH LIQUOR. The crystals are no longer fibers, and the composition is no longer solid.

FIG. 7 is an electron micrograph of an inventive example of sample prepared by adding an appropriate amount of acid converting agent (particles) to fiber mesh, maintaining the mesh microstructure (below) while allowing the creation of a low pH aqueous treatment liquor.

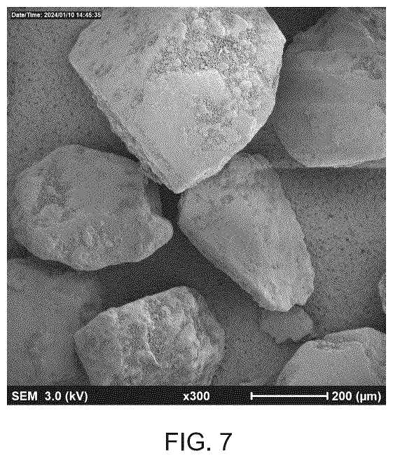

FIG. 8 is an electron micrograph of a comparative example of crystalline plates of potassium fatty acid carboxylate, that consequently do not form the required fiber mesh, illustrating the need for sodium fatty acid carboxylate in the invention.

FIG. 9 is an electron micrograph of a comparative example of crystalline plates of triethanolamine fatty acid carboxylate, that consequently do not form the required fiber mesh, illustrating the need for sodium fatty acid carboxylate in the invention.

### DETAILED DESCRIPTION OF THE INVENTION

The present invention is a solid-dissolvable composition comprising a solid-like base composed of sodium fatty acid carboxylate, water (generally contained within the sodium fatty acid carboxylate crystals), acid converting agent and an optimal benefit agent. The crystalline mesh (“mesh”) comprises a relatively rigid, three-dimensional, interlocking crystalline skeleton framework of fiber-like crystalline particles formed from crystallizing agents—a unit of which is the “substrate”. Water is used as a processing aid to facilitate the formation of the mesh microstructure, and generally non-bound water is removed after the formation of the mesh microstructure. The solid acid converting agent is added after the preparation of the mesh microstructure and removal of non-bound water. In non-limiting examples, the solid acid converting agent can be dispersed throughout the mesh, on top of the mesh or sandwiched between sheets of mesh. When combined in the right proportions, the mesh and acid converting agent create a low pH treatment liquor when added to water. An optional benefit agent can be added to the composition to impart special benefits to the composition, such as cleaning of clothes, deodorizing clothes in wash, conditioning of hair, or softening of skin. In non-limiting examples, the optional active agents can be dispersed throughout the mesh, on top of the mesh, or sandwiched between sheets of mesh.

The present invention may be understood more readily by reference to the following detailed description of illustrative compositions. It should be understood that the scope of the claims is not limited to the specific products, methods, conditions, devices, or parameters described herein, and that the terminology used herein is not intended to be limiting of the claimed invention.

“Solid-Dissolvable Composition,” a dry solid-dissolvable, single-measure composition comprising crystallizing agents of sodium fatty acid carboxylate which, when processed as described in the specification, form an interconnected crystalline mesh of fibers, at least one acid converting agent and optional benefit agents when added to water create a low pH aqueous treatment liquor.

The solid-dissolvable composition can take the form of a ‘sizeable’ particle, an agglomerate, a flake, a granule, a pellet, a tablet, a lozenge, a puck, a briquette, a brick, a solid block, a unit dose, or other solid form known to those of skill in the art. The solid-dissolvable composition does not take the form of powder. Herein, a ‘bead’ is a particular solid form, having a hemi-spherical shape with about a 2.5 mm radius.

“Mesh,” is an assembly of crystallizing agent fibers of sufficient concentration and orientation to create a rheological solid.

“Substrate” or “substrate mesh,” a ‘sizeable’ piece of material, composed primarily of a sodium fatty acid carboxylate mesh on which acid converting agent and/or benefit agent can be applied, or in which can be dispersed. In one preferred embodiment, the substrate has a density less than 1.0 grams per cubic centimeter, in another preferred embodiment the substrate has a density between about 1.0 grams per cubic centimeter and 0.45 grams per cubic centimeter, in another preferred embodiment the substrate has a density between about 0.95 grams per cubic centimeter and 0.50 grams per cubic centimeter, in another preferred embodiment the substrate has a density between about 0.95 grams per cubic centimeter and 0.60 grams per cubic centimeter, and in another preferred embodiment the substrate has a density between about 0.80 grams per cubic centimeters and 0.45 grams per cubic centimeters. One skilled in the art understands that the density can be determined by dividing the mass of the substrate by the volume of the substrate.

“Crystallizing agent,” is one or mixture of sodium fatty acid carboxylate with a plurality of chain lengths between C8-C12 which are solubilized in water above processing temperature and which forms crystals when cooled below the crystallization temperature, and in the absence of excess water.

“Fiber,” is a crystal of crystallizing agent that may have a minimum length of 10 μm and thickness of 2 um as determined by the FIBER TEST METHOD, with the general thread-like appearance.

“Acid converting agent,” is solid material that results in an acid solution (i.e., pH<7) when added to water, as determined by the PH TEST METHOD FOR ACID CONVERTING AGENT, and can be combined with at least one mesh to create a solid-dissolvable composition that results in a low pH aqueous treatment liquor when added with water.

“Optional benefit agent,” is a solid or liquid material that can be optionally added to the solid-dissolvable composition and adds additional benefits to the solid-dissolvable composition, such as, in non-limiting examples, for treating fabrics, skin, and hair.

“Low pH aqueous treatment liquor,” is a water in combination with a solid-dissolvable composition to create a low pH mixture (i.e., pH<7), as determined by the PH TEST METHOD FOR WASH LIQUOR.

The treatment liquor is used, in non-limiting examples, for treating fabrics, skin, and hair.

Not wishing to be limited to theory, it is believed that the mesh microstructure provides a solid-dissolvable consistent with the needs for a single-treatment consumer dose, the acid converting agent when combined in water with the mesh microstructure to create a low pH treatment liquor, whereas the solid-dissolvable composition is devoid of water to enable a separation of the two chemistries.

As used herein, the term “bio-based” material refers to a renewable material.

As used herein, the term “renewable material” refers to a material that is produced from a renewable resource. As used herein, the term “renewable resource” refers to a resource that is produced via a natural process at a rate comparable to its rate of consumption (e.g., within a 100-year time frame). The resource can be replenished naturally, or via agricultural techniques. Non-limiting examples of renewable resources include plants (e.g., sugar cane, beets, corn, potatoes, citrus fruit, woody plants, lignocellulose, hemicellulose, cellulosic waste), animals, fish, bacteria, fungi, and forestry products. These resources can be naturally occurring, hybrids, or genetically engineered organisms. Natural resources, such as crude oil, coal, natural gas, and peat, which take longer than 100 years to form, are not considered renewable resources. Because at least part of the material of the invention is derived from a renewable resource, which can sequester carbon dioxide, use of the material can reduce global warming potential and fossil fuel consumption.

As used herein, the term “bio-based content” refers to the amount of carbon from a renewable resource in a material as a percent of the weight (mass) of the total organic carbon in the material, as determined by ASTM D6866-10 Method B.

The term “solid” refers to the physical state of the composition under the expected conditions of storage and use of the solid-dissolvable composition.

As used herein, the articles including “a” and “an” when used in a claim, are understood to mean one or more of what is claimed or described.

As used herein, the terms “include,” “includes” and “including” are meant to be non-limiting.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Crystallizing Agent

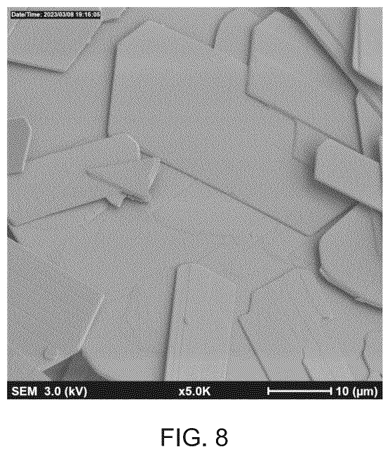

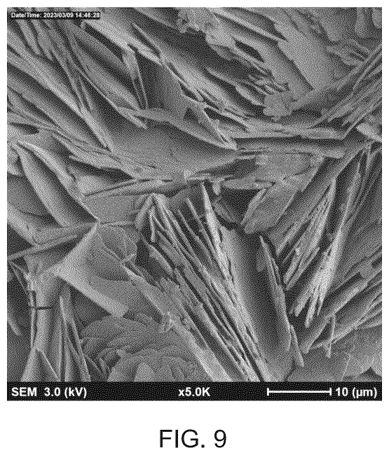

The crystallizing agents are selected from the small group sodium fatty carboxylates having primarily saturated chains and with chain lengths ranging from C8-C12, including sodium caprylic, sodium caprate, and sodium laurate. Surprisingly, when aqueous solutions of such sodium fatty acid carboxylates are crystallized as described in inventive examples here within, such sodium fatty acid carboxylates provide fiber crystals assembled into a fibrous mesh microstructure, ideal solubilization temperature for making and dissolution in use, and, by suitable blending, the resulting solid-dissolvable compositions have tunability in these properties for varied uses and conditions. In contrast, when aqueous solutions of such potassium fatty carboxylate (FIG. 8) and triethanolamine fatty carboxylate (FIG. 9) are crystallized as described in inventive examples here within, such crystals no longer form fibers and are unsuitable for this invention. The fiber mesh is also critical to ensure the solid-dissolvable composition is a rheological solid and most agreeable to single-measured dose for the consumer.

Preferred embodiments contain crystallizing agent crystals with greater than 60 wt. % sodium fatty acid carboxylates, more preferred embodiments contain greater than 70 wt. % sodium fatty acid carboxylates, more preferred embodiments contain greater than 80 wt. % sodium fatty acid carboxylates, more preferred embodiments contain greater than 90 wt. % sodium fatty acid carboxylates, and most preferred embodiments contain greater than 95 wt. % sodium fatty acid carboxylates, wherein the crystallizing agent crystals contain a plurality of fibers as determined by the FIBERS TEST METHOD

Preferred embodiments contain crystallizing agent crystals with greater than 80 wt. % C8, C10 and C12 sodium fatty acid carboxylates, more preferred embodiments contain crystallizing agent crystals with greater than 90 wt. % C8, C10 and C12, and most preferred embodiments contain crystallizing agent crystals with greater than 95 wt. % C8, C10 and C12. Only coincidental amounts of other chain length of crystallizing agent are included. One skilled in the art can re-apply the techniques described in the BLEND TEST METHOD (US2021/0320A1) to measure each chain length of sodium fatty acid carboxylates in substrate.

Non-limiting embodiments of solid-dissolvable compositions comprising less than 15 wt. % water preferably comprise less than about 90 wt. % crystallizing agent, more preferably comprise between 90 wt. % and 40 wt. % crystallizing agent, more preferably comprise between 90 wt. % and 50 wt. % crystallizing agent, more preferably comprise between 85 wt. % and 50 wt. % crystallizing agent, more preferably comprise between 85 wt. % and 60 wt. % crystallizing agent, most preferably comprise between 80 wt. % and 70 wt. % crystallizing agent.

### Water

The water is used as a processing aid to ensure the crystallizing agent forms fiber crystals on cooling. Most of the water is removed from the solid-dissolvable composition during processing where most remaining water is incorporated into the structure of the fiber crystals. Having water less than about 15 wt. %, more preferably less than 10 wt. % and most preferably less than 5 wt. % ensures that none of the acid converting agent is solubilized on the solid-dissolvable composition, to change the fiber microstructure of the crystals.

### Acid Converting Agent

The acid converting agents may be added to the solid-dissolvable composition to convert the otherwise basic treatment liquor into low pH treatment liquor, and is determined by the PH TEST METHOD FOR ACID CONVERTING AGENT.

Non-limiting examples of acid converting agents include adipic acid, aspartic acid, benzoic acid, carboxymethyloxymalonic acid, carboxymethyloxysuccinic acid, citric acid, formic acid, glycolic acid, benzoic acid, gluconic c acid, glutaric acid, hydroxyethyliminodiacetic acid, iminodiacetic acid, lactic acid, acetic acid, maleic acid, malic acid, malonic acid, oxydiacetic acid, oxydisuccinic acid, succinic acid, sulfamic acid, tartaric acid, tartaric acid, tartaric disuccinic acid, tartaric monosuccinic acid, citric acid, oxalic acid, salicylic acid, hyaluronic acid, mandelic acid, the salts of any of these acids, and mixtures thereof.

The acid converting agents may be a water-soluble or water-miscible acid. In some aspects, the acid converting agents have a solubility in water at 20° C. of at least about 10 g acid/100 ml water, or at least about 30 g acid/100 ml water, or at least about 50 g acid/100 ml water, or at least about 70 g acid/100 ml water, or at least about 85 g/100 ml water.

Non-limiting examples, preferred embodiments result in low pH aqueous treatment liquor with a pH between 2.0<pH<7.0, more preferred embodiments show a wash liquor between 3.0<pH<6.5 and most preferred embodiments show a wash liquor between 4.0<pH<6.3, using the PH TEST METHOD FOR WASH LIQUOR.

Preferred embodiments of acid converting agents are solid.

Preferred embodiments have critical relative humidity (CRH) for the mixtures of greater than 50% RH, more preferred embodiments have critical relative humidity for the mixtures of greater than 60% RH, more preferred embodiments have critical relative humidity for the mixtures of greater than 70% RH, more preferred embodiments have critical relative humidity for the mixtures of greater than 80% RH, and most preferred embodiments have critical relative humidity for the mixtures of greater than 90% RH. Not wishing to be bound by theory, but acid converting agents with higher critical relative humidity can be more easily transported within the supply chain to customers without the need for water-impermeable packaging.

### Benefit Agents

Benefit agents are optional, and used to impart additional benefits attained from the solid-dissolvable composition. It is understood the benefit agents described below may be used for other applications.

### Fabric Care Benefit Agents

Fabric care products are formulated to clean and potentially to enhance fabrics, where many treatments may benefit from low pH treatment liquors.

Non-limiting examples, benefit agents for adding freshness to fabrics include perfume, perfume capsules and pro-perfumes. Suitable benefit agents can be obtained from Givaudan Corp. of Mount Olive, New Jersey, USA, International Flavors & Fragrances Corp. of South Brunswick, New Jersey, USA, or Firmenich Company of Geneva, Switzerland or Milliken Company of Appleton, Wisconsin (USA). As used herein, a “perfume raw material” refers to one or more of the following ingredients: fragrant essential oils; aroma compounds; materials supplied with the fragrant essential oils, aroma compounds, stabilizers, diluents, processing agents, and contaminants; and any material that commonly accompanies fragrant essential oils, aroma compounds. Alternatively encapsulated perfume.

Non-limiting examples of perfume capsule wall materials include urea, silica, chitosan, and methyl formaldehyde.

The wall (or shell) material of the active agent delivery capsule may comprise: melamine, polyacrylamide, silicones, silica, polystyrene, polyurea, polyurethanes, polyacrylate based materials, polyacrylate esters based materials, gelatin, styrene malic anhydride, polyamides, aromatic alcohols, polyvinyl alcohol and mixtures thereof. The melamine wall material may comprise melamine crosslinked with formaldehyde, melamine-dimethoxyethanol crosslinked with formaldehyde, and mixtures thereof. The polystyrene wall material may comprise polystyrene cross-linked with divinylbenzene. The polyurea wall material may comprise urea crosslinked with formaldehyde, urea crosslinked with gluteraldehyde, polyisocyanate reacted with a polyamine, a polyamine reacted with an aldehyde and mixtures thereof. The polyacrylate based wall materials may comprise polyacrylate formed from methylmethacrylate/dimethylaminomethyl methacrylate, polyacrylate formed from amine acrylate and/or methacrylate and strong acid, polyacrylate formed from carboxylic acid acrylate and/or methacrylate monomer and strong base, polyacrylate formed from an amine acrylate and/or methacrylate monomer and a carboxylic acid acrylate and/or carboxylic acid methacrylate monomer, and mixtures thereof.

The polyacrylate ester-based wall materials may comprise polyacrylate esters formed by alkyl and/or glycidyl esters of acrylic acid and/or methacrylic acid, acrylic acid esters and/or methacrylic acid esters which carry hydroxyl and/or carboxy groups, and allylgluconamide, and mixtures thereof.

The aromatic alcohol-based wall material may comprise aryloxyalkanols, arylalkanols and oligoalkanolarylethers. It may also comprise aromatic compounds with at least one free hydroxyl-group, especially preferred at least two free hydroxy groups that are directly aromatically coupled, wherein it is especially preferred if at least two free hydroxy-groups are coupled directly to an aromatic ring, and more especially preferred, positioned relative to each other in meta position. It is preferred that the aromatic alcohols are selected from phenols, cresols (o-, m-, and p-cresol), naphthols (alpha and beta-naphthol) and thymol, as well as ethylphenols, propylphenols, fluorphenols and methoxyphenols.

The polyurea based wall material may comprise a polyisocyanate.

The polyvinyl alcohol-based wall may comprise e a crosslinked, hydrophobically modified polyvinyl alcohol, which comprises a crosslinking agent comprising i) a first dextran aldehyde having a molecular weight of from 2,000 to 50,000 Da; and ii) a second dextran aldehyde having a molecular weight of from greater than 50,000 to 2,000,000 Da.

The wall (shell) material nay comprise the reaction product of a biopolymer and a cross-linking agent.

The biopolymer may preferably be selected from the group consisting of a polysaccharide, a protein, a nucleic acid, a polyphenolic compound, derivatives thereof, and combinations thereof. Preferably, the biopolymer is selected from the group consisting of a polysaccharide selected from the group consisting of chitosan, starch, modified starch, dextran, maltodextrin, dextrin, cellulose, modified cellulose, hemicellulose, chitin, alginate, lignin, gum, pectin, fructan, carrageenan, agar, pullulan, suberin, cutin, cutan, melanin, silk fibronin, derivatives thereof, and combinations thereof.

In other embodiments, benefit agents that enhance freshness include various natural extracts and essences, which can comprise complex mixtures of ingredients, such as orange oil, lemon oil, rose extract, lavender, musk, patchouli, balsamic essence, sandalwood oil, pine oil, cedar, and the like.

Non-limiting examples, benefit agents for adding malodor benefits to fabric include malodor binding agents, including polymers that may include amine-based compounds, such as monoamines, amino acids, polyethyleneimine polymers (PEIs), modified PEIs, substituted PEIs; acrylic acid polymers, such as polyacrylate co-polymer, polyacrylic acid polymers, and modified acrylate copolymers; and modified methacrylate copolymers; or mixtures thereof. In other embodiments, malodor binding agents included solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof.

Non-limiting examples, benefit agents for adding fabric feel benefits to fabric include softening agent, including quaternary ammonium compound so that the particles can provide a softening benefit to laundered fabrics through the wash, and in particular during the wash sub-cycle of a washer having wash and rinse sub-cycles. The quaternary ammonium compound (quat) can be an ester quaternary ammonium compound. Suitable quaternary ammonium compounds include but are not limited to, materials selected from the group consisting of or selected from or selected from at least one of ester quats, amide quats, imidazoline quats, alkyl quats, amidoester quats and combinations thereof. Suitable ester quats include but are not limited to, materials selected from the group consisting of or selected from or selected from at least one of monoester quats, diester quats, triester quats and combinations thereof. Other examples include co-polymer of acrylic acid and diallyl dimethylammonium chloride such as Polyquaternium-22 and Merquat 280 (Lubrizol). Other examples include metathesized unsaturated polyol esters as found in U.S. Pat. No. 10,640,735 B2.

Non-limiting examples of benefit agents for adding softening benefits to fabric include fatty high melting point fatty compounds. The high melting point fatty compound useful herein has a melting point of 25° C. or higher, and is selected from the group consisting of fatty alcohols, fatty acids, fatty alcohol derivatives, fatty acid derivatives, and mixtures thereof. Suitable conditioning agents also include nonionic polymers and conditioning oils, such as hydrocarbon oils, polyolefins, and fatty esters.

Non-limiting examples of benefit agents for also adding softening benefits to fabric include silicone compounds, including silicone oils, cationic silicones, silicone gums, high refractive silicones, silicone resins, and organosilicones such as those found in U.S. Pat. No. 8,598,108 B2. Other embodiments include organic conditioning oils such as hydrocarbon oils, polyolefins, and fatty esters or combinations thereof, or those conditioning agents which otherwise form liquid, dispersed particles in the aqueous surfactant matrix herein. Non-limiting examples of benefit agents for bleaching fabric include bleaching agents, including non-limiting examples of peroxyacids such as phthalimidoperoxyhexanoic acid (PAP)), perborate, percarbonate, chlorine bleaches, oxygen bleaches, hypohalite bleaches, bleach precursors, bleach activators, bleach catalysts, hydrogen peroxide, bleach boosters, photobleaches, bleaching enzymes, free radical initiators, peroxygen bleaches, and mixtures thereof. Non-limiting examples of suitable bleaching agents include peroxyacids (for example phthalimidoperoxyhexanoic acid (PAP)), perborate, percarbonate, chlorine bleaches, oxygen bleaches, hypohalite bleaches, bleach precursors, bleach activators, bleach catalysts, hydrogen peroxide, bleach boosters, photobleaches, bleaching enzymes, free radical initiators, peroxygen bleaches, and mixtures thereof.

Non-limiting examples of benefit agents for cleaning fabric include cleaning enzyme agents including one or more enzymes that may be present in the fibrous elements and/or particles of the present invention. Non-limiting examples of suitable enzymes include proteases, amylases, lipases, cellulases, carbohydrases including man-nanases and endoglucanases, pectinases, hemicellulases, peroxidases, xylanases, phopholipases, esterases, cutinases, keratanases, reductases, oxidases, phenoloxidases, lipoxy-genases, ligninases, pullulanases, tannases, penosanases, malanases, glucanases, arabinosidases, hyaluraonidases, chrondroitinases, laccases, and mixtures thereof.

Non-limiting examples of benefit agents for brightening fabric include hueing agents, such as dyes, dye-clay conjugates, and pigments. Suitable dyes include small molecule dyes and polymeric dyes. Suitable small molecule dyes include small molecule dyes selected from the group consisting of: dyes falling into the Color Index (CI.) classifications of Direct Blue, Direct Red, Direct Violet, Acid Blue, Acid Red, Acid Violet, Basic Blue, Basic Violet and Basic Red, or mixtures thereof.

Non-limiting examples of benefit agents for affixing color on fabric include dye transfer inhibiting agents including polymeric dye transfer inhibiting agents such as polyvinylpyrrolidone polymers, polyamine N-oxide polymers, copolymers of N-vi-nylpyrrolidone and N-vinylimidazole, polyvinyloxazolidones and polyvinylimidazoles or mixtures thereof.

Non-limiting examples of benefit agents for removing microbes from fabric include one or more antimicrobials, antibacterials and antifungals. In one example, the antibacterial is chosen from triclosan, triclocarban, chlorohexidine, metronidazole and mixtures thereof. In another example, in addition to the antimicrobial active selected from polyvalent metal salts of pyrithione, the composition can further include one or more anti-fungal and/or anti-microbial actives. In another example, the anti-microbial active is selected from the group consisting of: coal tar, sulfur, azoles, selenium sulphide, particulate sulphur, keratolytic agents, charcoal, whitfield's ointment, castellani's paint, aluminum chloride, gentian violet, octopirox (piroctone olamine), ciclopirox olamine, undecylenic acid and its metal salts, potassium permanganate, selenium sul-phide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, ally-lamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-SO, Elestab HP-100, azelaic acid, lyticase, iodo-propynyl butylcarbamate (IPBC), isothiazalinones such as octyl isothiazalinone, and azoles, and mixtures thereof.

Non-limiting examples of benefit agents for suppressing suds while washing fabric include monocarboxylic fatty acid and soluble salts therein, high molecular weight hydrocarbons such as paraffin, fatty acid esters (e.g., fatty acid triglycerides), fatty acid esters of monovalent alcohols, aliphatic C18-C40 ketones (e.g., stearone), N-alkylated amino triazines, waxy hydrocarbons preferably having a melting point below about 1000 C., silicone suds suppressors, and secondary alcohols. Additional suitable antifoams are those derived from phenylpropylmethyl substituted polysiloxanes.

Non-limiting examples include natural extracts, which are complex, multi-component mixture obtained after using a solvent to select for, or remove, components of the herbal material. Extracts may be in dry, liquid, or semisolid form.

Non-limiting examples of benefit agent for suppressing malodor include bacterial endospore.

### Hair Care Benefit Agents

Hair care products are formulated to clean and potentially to enhance hair, where most shampoos require low pH treatment liquors. Benefit agents use to treat hair are known in the art.

Non-limiting examples of benefit agents for washing and removing solids from hair include sulfate-free anionic surfactants as a fraction or complete replacement of the sulfated surfactants. In non-limiting examples, sulfate-free surfactants include lauramido propyl hydroxy sultaine, sodium sodium lauroamphoacetate, cocoylglycinate, sodium methylcocoyltaurate, and decylglucoside. They may also include, for example, one or more compounds selected from taurates, monoalkylphosphates, dialkylphosphates, sarcosinates, sulfosuccinates, isethionates, and other taurates and mixtures thereof. They may also include, for example, one or more compounds selected from mild anionic amino acid-based surfactants such as sodium cocoylglycinate, sodium lauryl glycinate, sodium cocoyl alinate, glutamates, and mixtures thereof.

Non-limiting examples of benefit agents for conditioning hair include cationic surfactants as one cationic surfactant or a mixture of two or more cationic surfactants. Cationic surfactant can be selected from the group consisting of, but not limited to a mono-long alkyl quaternized ammonium salt; a combination of a mono-long alkyl quaternized ammonium salt and a di-long alkyl quaternized ammonium salt; a mono-long alkyl amine; a combination of a mono-long alkyl amine and a di-long alkyl quaternized ammonium salt; and a combination of a mono-long alkyl amine and a mono-long alkyl quaternized ammonium salt, a tertiary amine and combinations thereof. They may also include mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

Non-limiting examples of benefit agents for treating dandruff in the hair include anti-dandruff agents, non-limiting examples include selenium disulfide, pinonic acid, and/or zinc pyrithione.

Non-limiting examples of benefit agents for conditioning hair include silicones including dimethicone, amodimethicone, cyclopentasiloxane, cyclomethicone, dimethicone copolyols, and dimethiconol. Suitable silane-modified oils comprise a hydrocarbon chain selected from the group consisting of saturated oil, unsaturated oil, and mixtures thereof; and a hydrolysable silyl group covalently bonded to the hydrocarbon chain. Suitable silane-modified oils are described in detail in U.S. Application Ser. No. 61/821,818, filed May 10, 2013. Examples of suitable dimethicone co-polyols include, but are not limited to, PEG-i2 dimethicone, PEG/PPG-18/18 dimethicone, and PPG-12 dimethicone.

Non-limiting examples of benefit agents for conditioning hair include natural and/or essential oils including Eucalyptus globulus essential oil, clary sage essential oil, orange essential oil, tea tree essential oil, peppermint essential oil, rosemary essential oil, lemongrass essential oil and patchouli essential oil.

Non-limiting examples of benefit agents for conditioning hair also include cationic polymers including Polyquaternium-10, Polyquaternium-7, Polyquaternium-11, Guar hydroxypropyltrimonium chloride, cationic guar gum, Luviquat Care (Polyquaternium 44), a new branched copolymer of vinylpyrrolidone and quaternized vinylimidazolium salts.

Non-limiting examples of benefit agents for conditioning hair also include co-polymers including PVP (poly N-vinyl-2-pyrrolidone), PVA (polyvinyl acetate) and PVP/VA copolymer, Hydroxyethylcellulose, hydroxypropylmethylcellulose, carboxymethyl hydroxyethylcellulose, harboxymethyl hydroxypropyl guar and carbomer (poly acrylic acid, acrylates/C10-130 alkyl acrylate crosspolymer. EG-150 distearate, PEG-100 castor oil, PEG-100 lanoli and PEG-10 sorbitan laurate.

Non-limiting examples of benefit agents for conditioning hair also include gel networks made from high melting point fatty compound useful herein is selected from the group consisting of fatty alcohols, fatty acids, fatty alcohol derivatives, fatty acid derivatives, fatty amides, and mixtures thereof. Non-limiting example of fatty alcohols include, cetyl alcohol (having a melting point of about 56° C.), stearyl alcohol (having a melting point of about 58-59° C.), behenyl alcohol (having a melting point of about 71° C.), and mixtures thereof.

Non-limiting examples of benefit agents for removing oil from hair include absorbent for sebum including non-limiting examples of aluminum starch, corn starch, rice starch, rhassoul clay and silica.

Non-limiting examples of further benefit agents to treat hair include preservatives, perfumes or fragrances, coloring agents or dyes, conditioning agents, hair bleaching agents, thickeners, moisturizers, emollients, pharmaceutical actives, vitamins or nutrients, sunscreens, deodorants, sensates, plant extracts, nutrients, astringents, cosmetic particles, absorbent 55 particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne 60 agents, anti-microbial agents, sunscreens, tanning agents, exfoliation particles, hair growth or restorer agents, insect repellents, shaving lotion agents, co-solvents or other additional solvents, and similar other materials. Further non-limiting examples of optional ingredients include encapsulated perfumes, such as by 3-cyclodetrins, polymer microcapsules, starch encapsulated accords and combinations thereof.

### Skin Care Benefit Agents

Skin care products are formulated to clean and potentially to enhance skin and skin health, and products require low pH treatment liquors to match the natural pH of skin. Benefit agents use to treat skin are known in the art.

Non-limiting examples of benefit agents to treat skin include sugar amines, which are also known as amino sugars. Sugar amines refers to an amine derivative of a six-carbon sugar, with non-limiting examples including glucosamine, N-acetyl glucosamine, mannosamine, N-acetyl mannosamine, galactosamine, and N-acetylgalactosamine.

Non-limiting examples of further benefit agents to treat skin include the Vitamin B3 family. In one embodiment, the compositions of the present invention can contain a vitamin B compound. Vitamin B compounds are particularly useful for regulating skin condition as described in U.S. Pat. No. 5,939,082. The compositions of the present invention may include a safe and effective amount of a panthenoic acid derivative, including panthenol, dexpanthenol, ethyl panthenol, and mixtures thereof. These vitamin B5 compounds provide skin soothing, moisturizing, and anti-irritating benefits. The topical compositions of the present invention may comprise a safe and effective amount of one or more vitamin B6 compounds selected from the group consisting of pyridoxine, esters of pyridoxine (e.g., pyridoxine tripalmitate), amines of pyridoxine (e.g., pyridoxamine), salts of pyridoxine (e.g., pyridoxine HCl) and derivatives thereof, including pyridoxamine, pyridoxal, pyridoxal phosphate, pyridoxic acid, and mixtures thereof.

Non-limiting examples of further benefit agents to treat skin include the Vitamin C family. The compositions of the present invention may include a safe and effective amount of a compound from the Vitamin C family. Specifically, the compositions may include ascorbic acid and its salts, and ascorbic acid derivatives (e.g., magnesium ascorbyl phosphate, sodium ascorbyl phosphate, ascorbyl sorbate, ascorbylglucoside, and mixtures thereof). These anti-oxidant/radical scavengers are especially useful for providing protection against UV radiation which can cause increased scaling or texture changes in the stratum corneum and against other environmental agents which can cause skin damage.

Non-limiting examples of further benefit agents to treat skin include niacinamide (Vitamin B3), Vitamin C, collagen peptide, Vitamin E and alpha hydroxy acid (AHA), alone or in various combinations, thereof.

Non-limiting examples of further benefit agents to treat skin include natural extracts. The compositions of the present invention may include a safe and effective amount of extracts from natural products. Non-limiting examples include mulberry extract, placental extract, soy extract, green tea extract, and chamomile extract. These extracts provide a broad range of skin benefits such as anti-inflammatory, skin lightening, hair growth reduction and anti-irritancy.

Non-limiting examples of further benefit agents to treat skin include peptides. The compositions of the present invention may contain a safe and effective amount of a peptide, including but not limited to, di-, tri-, tetra-, penta-, hexa-peptides, and derivatives and mixtures thereof. As used herein, “peptide’ refers to peptides containing ten or fewer amino acids and their derivatives, isomers, and complexes with other species such as metal ions (e.g., copper, Zinc, manganese, magnesium, and the like). Skin care benefit agents may also include peptides alpha-hydroxy aldehydes and ketones. Examples include, but are not limited to, dihydroxyacetone, glyceraldehydes, 2.3-dihydroxy-succindialdehyde, 2,3 dimethoxysuccindialdehyde, erythrulose, erythrose, 2-amino-3-hydroxy-succindialdehyde and 3-benzylamino-3-hydroxy-succindialdehye. These compounds have a Sun-less tanning benefit when applied to skin.

Non-limiting examples of further benefit agents to treat skin include hexamidine and their salt. Preferably, the hexamidine is hexamidine isethionate.

Non-limiting examples of further benefit agents to treat skin include dehydroacetic acid and its salts, derivatives, or tautomers, thereof. These compounds are useful in (i) reducing sebum synthesis by the pilosebaceous glands, (ii) regulating the oily and/or shiny appearance of the skin, and (iii) treating acne and other related skin disorders in mammalian skin and scalp.

Non-limiting examples of further benefit agents to treat skin include water-soluble or water-swellable polymer. The polymers may be homopolymers, copolymers or a blend of polymers or copolymers. The polymers can be natural, synthetic, or semi-synthetic. Polymers can be straight chain or cross-linked. Polymers, containing either ionic and non-ionic groups, are contemplated. Ionic polymers include, but are not limited to, cationic, anionic, Zwitterionic, and amphoteric polymers. The polymers can be synthesized from a variety of monomers containing unsaturated groups or by synthetic mechanisms that result in a variety of linking groups, including polyurethanes, polyesters, polyamides, and polyureas in the polymer backbone.

Non-limiting examples of further benefit agents to treat skin include colorants, which provide color to a personal care product. The purpose of the colorant is to deliver the desirable shade or color to skin that the user is seeking as well as to even out skin tone by covering or hiding tonal imperfections. Useful colorants herein include water soluble dyes. Non-limiting examples of water-soluble acid dyes include D&C Red 33, FD&C Yellow No. 5, D&C Green No. 5, D&C Yellow No. 8, and D&C Yellow No. 10.

Non-limiting examples of further benefit agents to treat skin include fluid-absorbent particles. The fluid-absorbent particles can be any material that remains solid within the composition, including porous, hydrophilic, and solid particles. The fluid absorbent particles for use in the compositions of the present invention include moisture-absorbent materials such as silicas (or silicon dioxides), silicates, carbonates, various organic copolymers, and combinations thereof. Other fluid-absorbent particles suitable for use herein include kaolin, (hydrated aluminum silicates), mica, talc (hydrated magnesium silicates), starch or modified Starch, microcrystalline cellulose (e.g., Avicel from FMC Corporation), or other functionally similar fluid-absorbent polymer, any other silica-containing or non-silica-containing powder.

Non-limiting examples of further benefit agents to treat skin include Dialkylquates. Non-limiting examples include dialkyl dimethyl quaternaries (e.g., dialkyl (C-Cs) dimethyl ammonium chloride, ditallow dimethyl ammonium chloride, distearyl dimethyl ammonium methylsulfate) and imidazolinium quaternaries (e.g., methyl-1-oleyl amido ethyl-2-oleyl imidazolinium-methyl sulfate).

Non-limiting examples of further benefit agents to treat skin include ester oils. Non-limiting examples include the fatty acid mono and polyesters such as cetyl octanoate, octyl isonanoanate, myristyl lactate, cetyl lactate, isopropyl myristate, myristyl myristate, isopropyl palmitate, isopropyladipate, butyl Stearate, decyl oleate, cholesterol isostearate, glycerol monostearate, glycerol distearate, glycerol tristearate, alkyl lactate, alkyl citrate and alkyl tartrate. Sucrose ester and polyesters, Sorbitol ester, and the like. A second type of useful ester oil is predominantly comprised of triglycerides and modified triglycerides. These include vegetable oils such as jojoba, soybean, canola, Sunflower, safflower, rice bran, avocado, almond, olive, sesame, persic, castor, coconut, and mink oils. Synthetic triglycerides can also be employed provided they are liquid at room temperature. Modified triglycerides include materials such as ethoxylated and maleated triglyceride derivatives provided they are liquids. Proprietary ester blends such as those sold by Finetexas Finsolv are also Suitable, as is ethylhexanoic acid glyceride. A third type of ester oil is liquid polyester formed from the reaction of a dicarboxylic acid and a diol. Examples of polyesters suitable for the present invention are the polyesters marketed by ExxonMobil under the trade name PURESYNESTER®.

Non-limiting examples of further benefit agents to treat skin include silicone oils and waxes. The compositions of the present invention may include silicone oils and waxes. Silicone oils and waxes include poly dimethyl siloxane, organo functional silicones (alkyl and alkyl aryl, copolyol), and amino silicones.

Non-limiting examples of further benefit agents to treat skin include liquid fatty alcohols. The liquid fatty alcohols useful herein include those having from about 10 to about 30 carbon atoms. These liquid fatty alcohols may be straight or branched chain alcohols and may be saturated or unsaturated alcohols. Liquid fatty alcohols are those fatty alcohols which are liquid at 25° C. Nonlimiting examples of these compounds include oleyl alcohol, palmitoleic alcohol, isostearyl alcohol, isocetyl alcohol, and mixtures thereof. While poly fatty alcohols are useful herein, mono fatty alcohols are preferred. Non-limiting examples of these compounds also include triglycerides and modified triglycerides. These include vegetable oils such as jojoba, Soybean, canola, Sunflower, safflower, rice bran, avocado, almond, olive, Sesame, persic, castor, coconut, and mink oils. Non-limiting examples of these compounds also include ester lipids is liquid polyester formed from the reaction of a dicarboxylic acid and a diol.

Non-limiting examples of further benefit agents to treat skin include semi-solid hydrocarbons. These include linear and branched oils such as liquid paraffin, squalene, squalane, mineral oil, low viscosity synthetic hydrocarbons, petrolatum, linear and cyclic polydimethyl siloxane, organo functional silicones (alkyl and alkyl aryl), and aminosilicones, Suitable fatty acids include, for example, oleic acid, linoleic acid, isostearic acid, linolenic acid, ethyl linolenic acid, arachidonic acid, ricinolic acid, and mixtures thereof.

Non-limiting examples of further benefit agents to treat skin include preservatives.

Non-limiting examples of further benefit agents to treat skin include hyaluronic acid.

Non-limiting examples of further benefit agents to treat skin include fragrances in the form of neat perfumes, encapsulated perfumes and/or pro-perfumes.

Non-limiting examples of further benefit agents to treat skin include specific blends of benefit agents. A non-limiting example include dimethicone, niacinamide, lactic acid, polyacrylate crosspolymer-6, palmitoyl pentapeptide-4, 3-O-ethyl ascorbic acid, sodium hyaluronate, tocopheryl acetate, panthenol, trehalose, PEG-11 methyl ether dimethicone, sodium lactate, mica, titanium dioxide, sodium benzoate, and fragrance.

### Toilet Cleaning Benefit Agents

Toilet cleaning products are formulated to facilitate cleaning the inside of bowls, and products require low pH treatment liquors to remove rust, mineral deposits, fecal matter, and other non-organic materials. Benefit agents use to clean toilets are known in the art.

Non-limiting examples of benefit agents to treat toilets include a particulate foaming surfactant selected from the group consisting of linear C1-C12 alkylbenzene sulfonate, sodium C12-C16 alkyl methyl taurate, sodium alkylsulfate, C12-C18 alpha olefin sulfonate, disodium lauroyl or cocoyl glutamate, sulfonated C12-C16 alkyl polyglycosides (APG), lauryl sulfosuccinate, C12-C16 dimethyl amine oxide, lauramido propyl betaine, cocamido propyl betaine, lauramido propyl hydroxyl sultaine, cocamido propyl hydroxyl sultaine, and mixtures thereof.

Non-limiting examples of benefit agents to treat toilets further include an effervescent agents selected from the group consisting of sodium bicarbonate, sodium carbonate, and mixtures thereof; optionally combine with an effervescent activator, wherein the effervescent activator is selected from the group consisting of citric acid, tartaric acid, sulfamic acid, sulfuric acid on silica carrier, and mixtures thereof; combined with an effervescent protecting agent, for example wherein the effervescent protecting agent is selected from the group consisting of silica, zeolite, and mixtures thereof.

Non-limiting examples of benefit agents to treat toilets further include a cleaning active selected from the group consisting of perfumes, soil release polymers, enzymes, bacterial spores, chelants, antimicrobial compounds, bleaches, and mixtures, thereof.

Preferred embodiments of benefit agents are solid.

### Solid-Dissolvable Composition

As a non-limiting example, the solid-dissolvable composition contains a mesh of sodium fatty acid carboxylate fiber crystals as a substrate, in the form of a solid-like product, and at least one acid converting agent. In one embodiment, the acid converting agent is applied to one side of the mesh; in one embodiment, the acid converting agent is dispersed in the mesh; in one embodiment, the acid converting agent is ‘sandwiched’ between two meshes; in one embodiment, two acid converting agents are applied to the substrate mesh.

As a non-limiting example, the solid-dissolvable composition contains a mesh of sodium fatty acid carboxylate fiber crystals, in the form of a solid-like product, at least one acid converting agent, and at least one benefit agent. In one embodiment, the acid converting agent is applied to one side of the mesh and the benefit agent is applied to the opposite side; in one embodiment, the acid converting agent is applied to one side of the mesh and the benefit agent is applied to the same side; in one embodiment, the acid converting agent is dispersed in the mesh and the benefit applied to one surface of the mesh; in one embodiment, the acid converting agent is applied to the surface of the mesh and the benefit applied is dispersed within the mesh; in one embodiment, the acid converting agent and benefit agent are ‘sandwiched’ between two meshes.

As a non-limiting example, the solid-dissolvable composition contains two mesh of sodium fatty acid carboxylate fiber crystals, in the form of a solid-like product, at least one acid converting agent and at least one benefit agent. In one embodiment, the composition of one mesh of sodium fatty acid carboxylate fiber crystals hydrates quickly, one mesh of sodium fatty acid carboxylate fiber crystals hydrates slowly, with one acid converting agent. In one embodiment, the composition of one mesh of sodium fatty acid carboxylate fiber crystals hydrates quickly with one type of perfume capsule and one mesh of sodium fatty acid carboxylate fiber crystals hydrates slowly with another type of perfume capsule, with one acid converting agent.

As a non-limiting example, the solid-dissolvable composition contains one substrate mesh of sodium fatty acid carboxylate fiber crystals split open like ‘pita’ bread enabling to include at least one acid converting agent and at least one benefit agent in the pocket. In one embodiment, the composition of one substrate mesh of sodium fatty acid carboxylate as affixed to a second substrate mesh of sodium fatty acid carboxylate, entrapping at least one acid converting agent and at least one benefit agent in the pocket between. In one embodiment, water is used as a processing aid to affix the substrates to each other. In another embodiment, one of the substrates has greater than about 15% water and, as the water is removed, the acid converting agent and benefit agent are entrapped.

In one embodiment, solid-dissolvable compositions comprising less than 15 wt. % water preferably comprise less than about 90 wt. % crystallizing agent, more preferably comprise between 90 wt. % and 40 wt. % crystallizing agent, more preferably comprise between 90 wt. % and 50 wt. % crystallizing agent, more preferably comprise between 85 wt. % and 50 wt. % crystallizing agent, more preferably comprise between 85 wt. % and 60 wt. % crystallizing agent, most preferably comprise between 80 wt. % and 70 wt. % crystallizing agent.

In one embodiment, solid-dissolvable compositions comprising less than 15 wt. % water preferably comprise less than about 55 wt. % acid converting agent, more preferably comprise between 55 wt. % and 10 wt. % acid converting agent, more preferably comprise between 50 wt. % and 15 wt. % acid converting agent, more preferably comprise between 40 wt. % and 20 wt. % acid converting agent, most preferably comprise between 30 wt. % and 20 wt. % acid converting agent.

In one embodiment, solid-dissolvable composition comprises less than about 35 wt. %, less than about 25 wt. % benefit agent. In one embodiment, the benefit agent is used to treat hair; in one embodiment, the benefit agent is used to treat fabric; in one embodiment, the benefit agent is used to treat skin.

In one embodiment, the consumer product is added directly into the wash drum, at the start of the wash creating the low pH treatment liquor; in another embodiment, the consumer product is placed in the hand, water is added to the hand and product is lathered, and the low pH treatment liquor is applied to hair; in another embodiment, the consumer product is placed in the hand, water is added to the hand and product is creamed, and the low pH treatment liquor is applied to face or skin; in another embodiment, the consumer product is added to toilet water, dispersed and releases benefit agents to clean the toilet.

In one embodiment, the consumer product is sold in paper packaging, in one embodiment, the consumer product is sold in unit dose packaging; in one embodiment, the consumer product is sold with different colored substrates; in one embodiment, the consumer product is sold in a sachet; in one embodiment, the consumer product is sold in a recyclable container.

### Preparing Solid-Dissolvable Composition

In one non-limiting embodiment, a pre-mix is prepared by completely dissolving the crystallizing agent in water, cooling the mixture to form the fiber mesh, and drying the mesh below about 10 wt. % water to form a dry substrate. A solid-dissolvable composition is prepared by combining this mesh substrate with the acid converting agent, and with the optional benefit agent.

### Test Methods

### Fibers Test Method

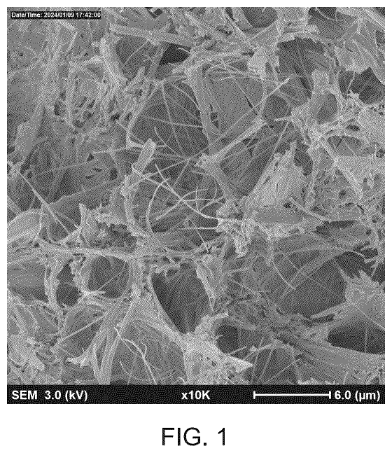

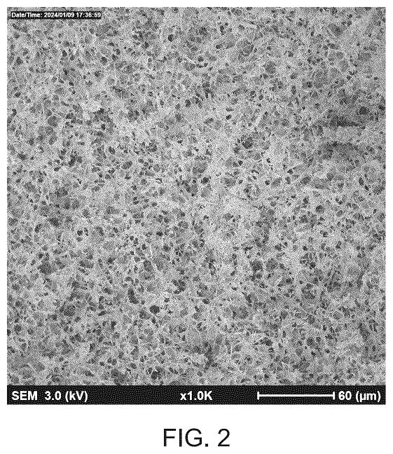

The Fiber Test Method is used to determine whether a solid dissolved composition crystallizes under process conditions and contains fiber crystals. A simple definition of a fiber is “a thread or a structure or an object resembling a thread”. Fibers have a long length in just one direction (e.g., FIG. 1 and FIG. 2). This differs from other crystal morphologies such as plates or platelets—with a long length in two or more directions (e.g., FIG. 8 and FIG. 9). Only solid dissolved compositions with fibers are in scope of this invention.

A sample measuring about 4 mm in diameter is mounted on an SEM specimen shuttle and stub (Quorum Technologies, AL200077B and E7406) with a slit precoated comprising a 1:1 mixture of Scigen Tissue Plus optimal cutting temperature (OCT) compound (Scigen 4586) compound and colloidal graphite (agar scientific G303E). The mounted sample is plunge-frozen in a liquid nitrogen-slush bath. Next, the frozen sample is inserted to a Quorum PP3010Tcryo-prep chamber (Quorum Technologies pp 3010T), or equivalent and allowed to equilibrate to −120° C. prior to freeze-fracturing. Freeze fracturing is performed by using a cold built-in knife in the cryo-prep chamber to break off the top of the vitreous sample. Additional sublimation is performed at −90° C. for 5 mins to eliminate residual ice on the surface of the sample. The sample is cooled further to −150° C. and sputter-coated with a layer of Pt residing in the cryo-prep chamber for 60 s to mitigate charging.

High resolution imaging is performed in a Hitachi Ethos NX5000 FIB-SEM (Hitachi NX5000), or equivalent.

To determine the fiber morphology of a sample, imaging is done at 20,000× magnification. At this magnification, individual crystals of the crystallizing agent may be observed. The magnification may be slightly adjusted to lower or higher values until individual crystals are observed. One skilled in the art can assess the longest dimension of the representative crystals in the image. If this longest dimension is about 10× or greater than the other orthogonal dimensions of the crystals, these crystals are considered fibers and in scope for the invention.

### Thermal Stability Test Method

All samples and procedures are maintained at room temperature (25°±3° C.) prior to testing, and at a relative humidity of 40±10% for 24 hours prior to testing.

In the Thermal Stability Test Method, differential scanning calorimetry (DSC) is performed on a 20 mg±10 mg specimen of sample composition. A simple scan is performed between 25° C. and at least 90° C., and the temperature at which the onset of the lowest-temperature peak (onset non-flat baseline) is observed to occur is reported as the Stability Temperature to the nearest ° C. For this instrumental configuration, negative values reflect adding power into the sample.

The sample is loaded into a DSC pan. All measurements are done in a high-volume-stainless-steel pan set (TA part #900825.902). The pan, lid and gasket are weighed and tared on a Mettler Toledo MT5 analytical microbalance (or equivalent; Mettler Toledo, LLC, Columbus, OH). The sample is loaded into the pan with a target weight of 20 mg (±/−10 mg) in accordance with manufacturer's specifications, taking care to ensure that the sample is in contact with the bottom of the pan. The pan is then sealed with a TA High Volume Die Set (TA part #901608.905). The final assembly is measured to obtain the sample weight. The sample is loaded into TA Q Series DSC (TA Instruments, New Castle, DE) in accordance with the manufacture instructions. The DSC procedure uses the following settings: 1) equilibrate at 25° C.; 2) mark end of cycle 1; 3) ramp 1.00° C./min to 90.00° C.; 4) mark end of cycle 3; then 5) end of method; Hit run.

### Humidity Test Method

The Water Vapor Sorption Test Method is used to determine the amount of water vapor sorption that occurs in a raw material or composition between being dried down at 0% RH and various RH at 25° C. In this method, 10 to 60 mg of sample are weighed, and the mass change associated with being conditioned with differing environmental states is captured in a dynamic vapor sorption instrument. The resulting mass gain is expressed as % change in mass per dried sample mass recorded at 0% RH.

This method makes use of a SPSx Vapor Sorption Analyzer with 1 ug resolution (ProUmid GmbH & Co. KG, Ulm, Germany), or equivalent dynamic vapor sorption (DVS) instrument capable of controlling percent relative humidity (% RH) to within ±3%, temperature to within ±2° C., and measuring mass to a precision of ±0.001 mg.

A 10-60 mg specimen of raw material or composition is dispersed evenly into a tared 1″ diameter Al pan. The Al pan on which raw material or composition specimen has been dispersed is placed in the DVS instrument with the DVS instrument set to 25° C. and 0% RH at which point masses are recorded˜every 15 minutes to a precision of 0.001 mg or better. After the specimen is in the DVS for a minimum of 12 hours at this environmental setting and constant weight has been achieved, the mass ma of the specimen is recorded to a precision of 0.01 mg or better. Upon completion of this step, the instrument is advanced in 10% RH increments up to 90% RH. The specimen is held in the DVS at each step for a minimum of 12 hours and until constant weight has been achieved, the mass mn of the specimen is recorded to a precision of 0.001 mg or better at each step.

For a particular specimen, constant weight can be defined as change in mass consecutive weighing that does not differ by more than 0.004%. For a particular specimen, % Change in mass per dried sample mass is defined as:

$$\% Change in mass per dried sample mass = \frac{{m}_{n} - {m}_{d}}{{m}_{d}} \times 100 \%$$

The % Change in mass per dried sample mass is reported in units of % to the nearest 0.01%

### pH Test Method for Acid Converting Agent

Water is taken from MilliQ water system with a measured impendence great than 16 mΩ.

Acid converting agents are prepared by taking 1.5 g±10 mg specimen of sample composition, adding to 100 g±1 g water, and stirring at minimum 250 RPM for 1 minute before measuring pH following the ASTM E70-19: Standard Test Method for pH of Aqueous Solutions With the Glass Electrode is used to measure pH. Non-limiting examples, preferred embodiments show an acid wash liquor pH<7.0.

### pH Test Method for Wash Liquor

Water is taken from MilliQ water system with a measured impendence great than 16 mΩ.

A sample of crystallizing agents or Solid-Dissolvable Composition is prepared by taking minimum 1.5 g±10 mg specimen of sample composition, adding to 100 g±1 g water, and stirring at minimum 250 RPM on a 70° C. hotplate for 10 minutes. Samples are then let to stand at room temperature for 10 minutes prior to measuring pH following the ASTM E70-19: Standard Test Method for pH of Aqueous Solutions With the Glass Electrode is used to measure pH.

The method returns the measure pH for inventive compositions and returns the value ‘NS’ for those compositions not soft enough to measure and returns ‘NM’ for those compositions that are not solid and/or lacks uniformity in fibers.

### Moisture Test Method

To measure the moisture content of the sample, measurements are made using a Mettler Toledo V30S Volumetric KF Titrator. The instrument uses Honeywell Fluka Hydranal Solvent (cat. #34800-1L-US) to dissolve the sample, Honeywell Fluka Hydranal Titrant-5 (cat. #34801-1L-US) to titrate the sample and is equipped with three drying tubes (Titrant Bottle, Solvent Bottle, and Waste Bottle) packed with Honeywell Fluka Hydranal Molecular sieve 3 nm (cat. #34241-250g) to preserve the efficacy of the anhydrous materials. The method used to measure the sample is Type “KF vol,” ID “U8000,” and Title “KFVol 2-comp 5,” and has eight lines which are each method functions.

The Line 1, Title has the following things selected: the Type is set to Karl Fischer titration Vol.; Compatible with is set to be V10S/V20S/V30S/T5/T7/T9; ID is set as U8000; Title is set as KFVol 2-comp 5; Author is set as Administrator; the Date/Time along with the Modified on and Modified by were defined by when the method was created; Protect is set to no; and SOP is set to None.

The Line 2, Sample has two options, Sample and Concentration. When the Sample option is chosen, the following fields are defined as: Number of IDs is set as 1; ID 1 is set as—; Entry type is selected to be Weight; Lower limit is set as 0.0 g; the Upper limit is set as 5.0 g; Density is set as 1.0 g/mL; Correction factor is set as 1.0; Temperature is set to 25.0° C.; Autostart is selected; and Entry is set to After addition. When the Concentration option is chosen, the following fields are defined as: Titrant is selected as KF 2-comp 5; Nominal conc. is set as 5 mg/mL; Standard is selected to be Water-Standard 10.0; Entry type is selected to be Weight; Lower limit is set as 0.0 g; Upper limit is set as 2.0 g; Temperature is set as 25.0° C.; Mix time is set as 10 s; Autostart is selected; Entry is selected to be After addition; Conc. lower limit is set to be 4.5 mg/mL; and Conc. upper limit is set to be 5.6 mg/mL.

The Line 3, Titration stand (KF stand) has the following fields defined as: Type is set to KF stand; Titration stand is selected to be KF stand; Source for drift is selected to be Online; Max. start drift is set to be 25.0 μg/min.

The Line 4, Mix time has the following fields defined as: Duration is set to be 150 s.

The Line 5, Titration (KF Vol) [1] has six options, Titrant, Sensor, Stir, Predispense, Control, and Termination. When the Titrant option is chosen, the following fields are defined as: Titrant is selected to be KF 2-comp 5; Nominal conc. is set to be 5 mg/mL; and Reagent type is set as 2-comp. When the Sensor option is chosen, the following fields are defined as: Type is set to Polarized; Sensor is selected as DM143-SC; Unit is set as mV; Indication is set as Voltametric; and Ipol is set as 24.0 μA. When the Stir option is chosen, the following fields are defined as: Speed is set as 50%. When the Pre-dispense option is chosen, the following fields are defined as: Mode is selected to be None; Wait time is set to be Os. When the Control option is chosen, the following fields are defined as: End Point is set to 100.00 mV; Control band is set to be 400.00 mV; Dosing rate (max) is set to be 3 mL/min; Dosing rate (min) is set to be 100 μL/min; and Start is selected to be Normal. When the Termination option is chosen, the following fields are defined as: Type is selected as Drift stop relative; Drift is set to 15.0 μg/min; At Vmax 15 mL; Min. time is set as 0 s; and Max. time is set as co s.

The Line 6, Calculation has the following fields defined as: Result type is selected to be Predefined; Result is set as Content; Result unit is set as %; Formula is set as R1=(VEQ*CONC−TIME*D . . . ); Constant C=is set as 0.1; Decimal places is set as 2; Result limits is not selected; Record statistics is selected; Extra statistical functions is not selected.

The Line 7, Record has the following fields defined as: Summary is selected to be Per sample; Results is selected to be No; Raw results is selected to be No; TABLE of meas. values is selected to be No; Sample data is selected to be No; Resource data is selected to be No; E-V is selected to be No; E-t is selected to be No; V-t is selected to be No; H2O-tis selected to be No; Drift-t is selected to be No; H2O-t & Drift-t is selected to be no; V-t & Drift-t is selected to be No; Method is selected to be No; and Series data is selected to be No.

The Line 8, End of Sample has the following fields defined as: Open series is selected.

Once the method is selected, press Start, the following fields are defined as: Type is set as Method; Method ID is set as U8000; Number of samples is set as 1; ID 1 is set as—; and Sample size is set as 0 g. The Start option is the pressed again. The instrument will measure the Max Drift, and once it reaches a steady state will allow the user to select Add sample, at which point the user will add the Three-hole adapter and stoppers are removed, the sample is loaded into the Titration beaker, the Three-hole adapter and stoppers are replaced, and the mass, g, of the sample is entered into the Touchscreen. The reported value will be the weight percent of water in the sample. This measure is repeated in triplicate for each sample, and the average of the three measures is reported.

### EXAMPLES

Inventive solid-dissolvable composition comprising a crystallizing agent in the form fiber mesh microstructure, an acid converting agent, less than 10 wt. % water, and an optionally a benefit agent. When added to water, the composition creates a low pH aqueous treatment liquor preferably between 2.0<pH<7.0 as measured by the PH TEST METHOD FOR WASH LIQUOR.

### Materials

(1) Water: Millipore, Burlington, MA (18 m-ohm resistance)
(2) Fatty acid blend: C810L, Procter & Gamble Chemicals, Sample Code: SR26399
(3) Fatty acid (lauric acid): Peter Cremer, Cat. #FA-1299 Lauric Acid
(4) Sodium hydroxide (50 wt. % solution): Fisher Scientific, Cat. #SS254-4
(5) Citric acid: Archer Daniels Midland, Product Code #020420
(6) Sodium citrate: Sodium citrate: Jungbunzlauer, Material #131620
(7) Sodium caprylic (sodium octanoate, NaC8): TCI Chemicals, Cat #00034
(8) Sodium caprate (sodium decanoate, NaC10): TCI Chemicals, Cat #D0024
(9) Sodium laurate (sodium dodecanoate, NaC12): TCI Chemicals, Cat #L0016
(10) Succinic acid: Sigma-Aldrich, Cat #398055-500G
(11) Mandelic acid: Sigma-Aldrich, Cat #8.06913.0250
(12) Oxalic acid: Sigma-Aldrich, Cat #241172-50G
(13) Malic acid: Sigma-Aldrich, Cat #M0875-500G
(14) N-hance 3271: Ashland Specialty Ingredients, Material #791483
(15) Octopirox: Clariant, Material #DAFA150950
(16) Niacinamide: DSM Nutritional Products, Product Code 0487848

### Example 1

### Discussion

Inventive examples demonstrate solid-dissolvable compositions which are composed of a substrate mesh comprising an inventive blend of sodium fatty acid carboxylates and of an acid converting agent, that may be added into the drum of a washer machine at the start of a wash cycle to create a low pH aqueous treatment liquor to impart benefits. In a non-limiting case, such compositions may also include Fabric Care Benefit Agents.

Example AA shows a comparative case, where the use of the solid dissolvable composition in the absence of acid converting agent results in a high pH aqueous treatment liquor with a pH>7. This also demonstrates that the sodium fatty acid carboxylates used to create the substrate mesh fiber microstructure are inherently basic when added to water, and are alone (without an acid converting agent) unsuitable for this invention.

Example AB shows a comparative case, where the solid-dissolvable composition has a percent acid converting agent <10 wt. % and does not create a low pH aqueous treatment liquor with a pH<7, when mixed with water.

Example AC—Examples AH show inventive solid-dissolvable compositions that create low pH aqueous treatment liquor with a pH<7, when mixed with water. The compositions are prepared with the percent of acid converting agent in the solid-dissolvable composition between about 10 wt. % and 50 wt. %, whereas the higher percentage of acid converting agent the lower the pH of the treatment liquor.

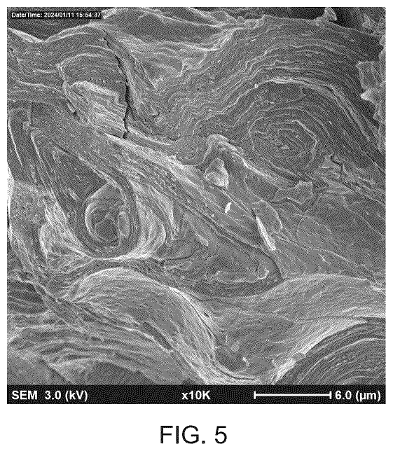

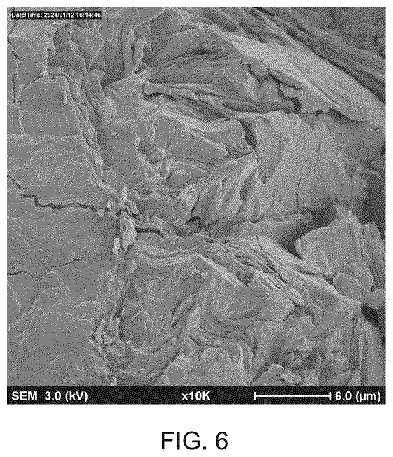

Example AI shows a comparative example in which the amount of water in the solid-dissolvable composition greater than 5 wt. % results in the degradation of the fiber mesh microstructure, and such compositions with higher water levels are unsuitable for this invention. Not wishing to be bound by theory, it is believed that combining the substrate mesh with acid converting agent in the presence of significant amount of unbound water allows conversion of the fibers into non-fibers (FIG. 5 and FIG. 6) resulting in a ‘mushy’ product.

### Preparation of Examples

The pre-mix is prepared by melting fatty acid in combination with water to 60° C., and then neutralizing the combination with NaOH solution. The entire mixture is stirred until it becomes completely clear, and poured into a rectangular mold and allowed to cool at 4° C. for about 8 hours. The composition solidifies into a sheet of substrate mesh. Water is deliberately removed by passing large amounts of air over the surface of the substrate mesh.

The solid-dissolvable composition is prepared for Example AA-AI, by removing the sheet of substrate mesh from the mold and then dried to less than about 5 wt. % as determined by the MOISTURE TEST METHOD. It is then cut into sections of substrate which are about 1.5 g to 3 cm×2.5 cm×3 mm. The acid converting agent is added to the top of the substrate mesh.

Example AH is prepared by removing the sheet of substrate mesh from the mold and adding to the acid converting agent to the top of the substrate mesh, is the absence of any deliberate drying.

As shown in Tables 1, 2, and 3, using the PH TEST METHOD FOR WASH LIQUOR, the Comparative Examples have a pH that is unmeasurable or greater than 7.0 and the Inventive Examples have a pH less than 7.0.

TABLE 1

| | | | |
|---|---|---|---|
| Sample | Sample | Sample | Sample |
| AA | AB | AC | AD |
| Comparative | Comparative | Inventive | Inventive |

Pre-Mix

| | | | | | |
|---|---|---|---|---|---|
| (1) Water | 99.9 g | 100.3 | g | 99.9 g | 99.9 g |
| (2) C8C10L | 13.7 g | 13.7 | g | 13.7 g | 13.7 g |
| (3) Lauric | 21.1 g | 21.1 | g | 21.1 g | 21.1 g |
| Acid | | | | | |
| (4) NaOH | 15.5 g | 15.5 | g | 15.5 g | 15.5 g |

Solid-Dissolvable Composition

| Substrate | 1.50 g | 1.51 | g | 1.50 g | | 2.99 g | |
|---|---|---|---|---|---|---|---|
| Mesh | | | | | | | |
| (5) Acid | 0 | 0.10 | g | 0.48 g | | 0.95 g | |
| converting | | | | | | | |
| agent | | | | | | | |
| Water | <5% | <5% | | <5% | | <5% | |
| % Acid | 0 wt. % | 6.2 | wt. % | 24.1 | wt. % | 24.0 | wt. % |
| converting | | | | | | | |
| agent | | | | | | | |
| % Benefit | 0 wt. % | 0 | wt. % | 0 | wt. % | 0 | wt. % |
| agent | | | | | | | |

Performance

| | | | | |
|---|---|---|---|---|
| pH | 9.44 | 8.18 | 5.29 | 5.01 |

TABLE 2

| | | | |
|---|---|---|---|
| Sample | Sample | Sample | Sample |
| AE | AF | AG | AH |
| Inventive | Inventive | Inventive | Inventive |

Pre-Mix

| | | | | |
|---|---|---|---|---|
| (1) Water | 99.9 g | 99.9 g | 99.9 g | 99.9 g |
| (2) C8C10L | 13.7 g | 13.7 g | 13.7 g | 13.7 g |
| (3) Lauric | 21.1 g | 21.1 g | 21.1 g | 21.1 g |
| Acid | | | | |
| (4) NaOH | 15.5 g | 15.5 g | 15.5 g | 15.5 g |

Solid-Dissolvable Composition

| Substrate | 3.01 g | | 1.50 g | | 1.50 g | | 1.50 g | |
|---|---|---|---|---|---|---|---|---|
| Mesh | | | | | | | | |
| (5) Acid | 0.47 g | | 0.22 g | | 1.50 g | | 0.39 g | |
| converting | | | | | | | | |
| agent | | | | | | | | |
| Water | <5 | wt. % | <5 | wt. % | <5 | wt. % | <5 | wt. % |
| % Acid | 13.6 | wt. % | 12.9 | wt. % | 50.0 | wt. % | 20.6 | wt. % |
| converting | | | | | | | | |
| agent | | | | | | | | |
| % Benefit | 0 | wt. % | 0 | wt. % | 0 | wt. % | 0 | wt. % |
| agent | | | | | | | | |

Performance

| | | | | |
|---|---|---|---|---|
| pH | 6.31 | 6.54 | 3.45 | 5.30 |

TABLE 3

Sample AI
Comparative

Pre-Mix

| | | |
|---|---|---|
| (1) Water | 10.1 | g |
| (2) C8C10L | 1.4 | g |
| (3) Lauric Acid | 2.1 | g |
| (4) NaOH | 1.6 | g |

Solid-Dissolvable Composition

| | | |
|---|---|---|
| Substrate Mesh | 0.42 | g |
| (5) Acid converting agent | 0.14 | g |
| Water | 18.8 | wt. % |
| % Acid converting agent | 25.0 | wt. % |
| % Benefit agent | 0 | wt. % |

Performance

| | |
|---|---|
| pH | NM |

### Example 2

### Discussion

Inventive examples demonstrate solid-dissolvable compositions is composed of mesh from a blend of sodium fatty acid carboxylates, combined with a physical mixtures of citric acid converting agent, which result in a low pH aqueous treatment liquor when combined with water. In this example, the mesh is prepared by previously prepared sodium fatty acid carboxylates.

Example BA—Examples BB are comparative examples of solid-dissolvable compositions that create high pH aqueous treatment liquor with a pH >7, when mixed with water. The weight percent of acid converting agent is less than 10 wt. %, making these compositions unsuited for this invention.

Example BC—Examples BH are inventive examples of solid-dissolvable compositions that do not create low pH aqueous treatment liquor with a pH<7, when mixed with water. The weight percent of acid converting agent is greater than 10 wt. %, making these compositions suited for this invention.

### Preparation

The pre-mix is prepared by melting fatty acid in combination with water to 60° C., and then neutralizing the combination with NaOH solution. The entire mixture is stirred until it becomes completely clear, and poured into a rectangular mold and allowed to cool at 4° C. for about 8 hours. The composition solidifies into a sheet of substrate mesh.

The solid-dissolvable composition is prepared for Example BA-BH, by removing the sheet of substrate mesh from the mold and then dried to less than about 5 wt. % as determined by the MOISTURE TEST METHOD. It is then cut into sections of substrate which are about 1.5 g to 3 cm×2.5 cm×3 mm. The acid converting agent is added to the top of the substrate mesh.

The performance is determined by the PH TEST METHOD FOR WASH LIQUOR.

TABLE 4

| | | | |
|---|---|---|---|
| | | Sample | Sample |
| Sample BA | Sample BB | BC | BD |
| Comparative | Comparative | Inventive | Inventive |

Pre-Mix

| (1) Water | 35.1 | g | 35.3 | g | 35.1 | g | 35.1 | g |
|---|---|---|---|---|---|---|---|---|
| (7) NaC8 | — | | 6.0 | g | — | | 0 | |
| (8) NaC10 | 6.0 | g | — | | 6.0 | g | 6.0 | g |
| (9) NaC12 | 9.0 | g | 9.0 | g | 9.0 | g | 9.0 | g |

Solid-Dissolvable Composition

| Substrate | 1.50 | | 1.51 | g | 1.50 | g | 3.00 | g |
|---|---|---|---|---|---|---|---|---|
| Mesh | | | | | | | | |
| (5) Acid | — | | 0.10 | g | 0.48 | g | 0.95 | g |
| converting | | | | | | | | |
| agent | | | | | | | | |
| Water | <5 | wt. % | <5 | wt. % | <5 | wt. % | <5 | wt. % |
| % Acid | 0 | wt. % | 6.0 | wt. % | 24.1 | wt. % | 24.0 | wt. % |
| converting | | | | | | | | |
| agent | | | | | | | | |
| % Benefit | 0 | wt. % | 0 | wt. % | 0 | wt. % | 0 | wt. % |
| agent | | | | | | | | |

Performance

| | | | | |
|---|---|---|---|---|
| pH | 8.77 | 8.41 | 5.86 | 5.69 |

TABLE 5

| | | | |
|---|---|---|---|
| Sample | Sample | Sample | Sample |
| BE | BF | BG | BH |
| Inventive | Inventive | Inventive | Inventive |

Pre-Mix

| (1) Water | 35.1 | g | 35.1 | g | 35.1 | g | 35.1 | g |
|---|---|---|---|---|---|---|---|---|
| (7) NaC8 | — | | — | | — | | — | |
| (8) NaC10 | 6.0 | g | 6.0 | g | 6.0 | g | 6.0 | g |
| (9) NaC12 | 9.0 | g | 9.0 | g | 9.0 | g | 9.0 | g |

Solid-Dissolvable Composition

| Substrate | 3.00 | g | 1.50 | g | 1.51 | g | 1.50 | g |
|---|---|---|---|---|---|---|---|---|
| Mesh | | | | | | | | |
| (5) Acid | 0.48 | g | 0.23 | g | 1.50 | g | 0.39 | g |
| converting | | | | | | | | |
| agent | | | | | | | | |
| Water | <5 | wt. % | <5 | wt. % | <5 | wt. % | <5 | wt. % |
| % Acid | 13.7 | wt. % | 13.0 | wt. % | 49.8 | wt. % | 20.8 | wt. % |
| converting | | | | | | | | |
| agent | | | | | | | | |
| % Benefit | 0 | wt. % | 0 | wt. % | 0 | wt. % | 0 | wt. % |
| agent | | | | | | | | |

Performance

| | | | | |
|---|---|---|---|---|
| pH | 6.77 | 6.67 | 3.40 | 6.23 |

TABLE 6

| | |
|---|---|
| Sample BI | Sample BJ |
| Inventive | Inventive |

Pre-Mix

| | | | | |
|---|---|---|---|---|
| (1) Water | 35.1 | g | 35.1 | g |

| | | |
|---|---|---|
| (7) NaC8 | — | — |

| | | | | |
|---|---|---|---|---|
| (8) NaC10 | 6.0 | g | 6.0 | g |
| (9) NaC12 | 9.0 | g | 9.0 | g |

Solid-Dissolvable Composition

| | | | | |
|---|---|---|---|---|
| Substrate Mesh | 1.50 | g | 1.51 | g |
| (5) Acid converting agent | 1.82 | g | 0.48 | g |
| Water | <5 | wt. % | <5 | wt. % |
| % Acid converting agent | 54.8 | wt. % | 24.0 | wt. % |
| % Benefit agent | 0 | wt. % | 0 | wt. % |

Performance

| | | |
|---|---|---|
| pH | 3.35 | 5.58 |

### Example 3

### Discussion

Inventive examples demonstrate solid-dissolvable compositions is composed of mesh from a blend of sodium fatty acid carboxylates, combined with a physical mixtures of different acid converting agent, which result in a low pH aqueous treatment liquor when combined with water.

Example CA—Example CG demonstrate the pH of the low pH treatment liquor may be varied by adjusting the acid converting agent, even ostensibly the weight percent of the acid converting acids is the same (i.e., 24.1 wt. %).

Example CE demonstrate the pH of the low pH treatment liquor may be adjusted also by the weight percent of the acid converting agent, relative to Example CC.

### Preparation

The pre-mix is prepared by melting fatty acid in combination with water to 60° C., and then neutralizing the combination with NaOH solution. The entire mixture is stirred until it becomes completely clear, and poured into a rectangular mold and allowed to cool at 4° C. for about 8 hours. The composition solidifies into a sheet of substrate mesh.

The solid-dissolvable composition is prepared for Example CA-CE, by removing the sheet of substrate mesh from the mold and then dried to less than about 5 wt. % as determined by the MOISTURE TEST METHOD. It is then cut into sections of substrate which are about 1.5 g to 3 cm×2.5 cm×3 mm. The acid converting agent is added to the top of the substrate mesh.

The performance is determined by the PH TEST METHOD FOR WASH LIQUOR.

TABLE 7

| | | |
|---|---|---|
| Sample | Sample | Sample |
| CA | CB | CC |
| Inventive | Inventive | Inventive |

Pre-Mix

| (1) Water | 35.0 | g | 35.0 | g | 35.0 | g |
|---|---|---|---|---|---|---|
| (7) NaC8 | — | | — | | — | |
| (8) NaC10 | 6.0 | g | 6.0 | g | 6.0 | g |
| (9) NaC12 | 9.0 | g | 9.0 | g | 9.0 | g |

Solid-Dissolvable Composition

| Substrate Mesh | 1.50 | g | 1.50 | g | 1.49 | g |
|---|---|---|---|---|---|---|
| (10) Acid converting agent | 0.48 | g | — | | — | |
| (11) Acid converting agent | — | | 0.48 | g | — | |
| (12) Acid converting agent | — | | — | | 0.48 | g |
| (13) Acid converting agent | — | | — | | — | |
| Water | <5 | wt. % | <5 | wt. % | <5 | wt. % |
| % Acid converting agent | 24.1 | wt. % | 24.1 | wt. % | 24.3 | wt. % |
| % Benefit agent | 0 | wt. % | 0 | wt. % | 0 | wt. % |

Performance

| | | | |
|---|---|---|---|
| pH | 5.64 | 6.80 | 3.79 |

TABLE 8

| | | | |
|---|---|---|---|
| Sample | Sample | Sample | Sample |
| CD | CE | CF | CG |
| Inventive | Inventive | Inventive | Inventive |

Pre-Mix

| | | | | | | | | |
|---|---|---|---|---|---|---|---|---|
| (1) Water | 100.3 | g | 100.3 | g | 100.3 | g | 100.3 | g |
| (2) C8C10L | 13.7 | g | 13.7 | g | 13.7 | g | 13.7 | g |
| (3) Lauric Acid | 21.1 | g | 21.1 | g | 21.1 | g | 21.1 | g |
| (4) NaOH | 15.5 | g | 15.5 | g | 15.5 | g | 15.5 | g |

Solid-Dissolvable Composition

| Substrate Mesh | 1.50 | g | 1.50 | g | 1.50 | g | 1.50 | g |
|---|---|---|---|---|---|---|---|---|
| (5) Acid | | | | | 0.24 | g | 0.24 | g |
| converting | | | | | | | | |
| agent | | | | | | | | |
| (6) Acid | — | | — | | 0.24 | g | — | |
| converting | | | | | | | | |
| agent | | | | | | | | |
| (11) Acid | — | | — | | — | | 0.24 | g |
| converting | | | | | | | | |
| agent | | | | | | | | |
| (12) Acid | 0.17 | g | — | | — | | — | |
| converting | | | | | | | | |
| agent | | | | | | | | |
| (13) Acid | — | | 0.47 | g | — | | — | |
| converting | | | | | | | | |
| agent | | | | | | | | |
| Water | <5 | wt. % | <5 | wt. % | <5 | wt. % | <5 | wt. % |
| % Acid | 10.2 | wt. % | 24.0 | wt. % | 24.5 | wt. % | 24.3 | wt. % |
| converting | | | | | | | | |
| agent | | | | | | | | |
| % Benefit | 0 | wt. % | 0 | wt. % | 0 | wt. % | 0 | wt. % |
| agent | | | | | | | | |

Performance

| | | | | |
|---|---|---|---|---|
| pH | 6.23 | 4.97 | 6.35 | 5.99 |

### Example 4

### Discussion

Inventive examples demonstrate solid-dissolvable compositions is composed of mesh from a blend of sodium fatty acid carboxylates, combined with mixtures of different acid converting agents and benefit agents, in a single composition, which results in a low pH aqueous treatment liquor when combined with water.

### Preparation

The pre-mix is prepared by combining pre-neutralized sodium fatty acid carboxylate with water. The entire mixture is heated at 60f° C. stirred until it becomes completely clear.

The solid-dissolvable composition is prepared for Example DA-DC, by removing the sheet of substrate mesh from the mold and then dried to less than about 5 wt. % as determined by the MOISTURE TEST METHOD. It is then cut into sections of substrate which are about 1.5 g to 3 cm×2.5 cm×3 mm. The acid converting agent and benefit agent are added to the top of the substrate mesh.

The performance is determined by the PH TEST METHOD FOR WASH LIQUOR.

TABLE 9

| | | |
|---|---|---|
| Sample | Sample | Sample |
| DA | DB | DC |
| Inventive | Inventive | Inventive |

Pre-Mix

| | | | | | | |
|---|---|---|---|---|---|---|
| (1) Water | 100.3 | g | 100.3 | g | 100.3 | g |
| (2) C8C10L | 13.7 | g | 13.7 | g | 13.7 | g |
| (3) Lauric Acid | 21.2 | g | 21.1 | g | 21.1 | g |
| (4) NaOH | 15.5 | g | 15.5 | g | 15.5 | g |

Solid-Dissolvable Composition

| Substrate Mesh | 1.50 | g | 1.50 | g | 1.51 | g |
|---|---|---|---|---|---|---|
| (5) Acid converting agent | 0.48 | g | 0.48 | g | — | |
| (11) Acid converting agent | — | | — | | 0.47 | g |
| (14) Benefit agent | 0.14 | g | — | | — | |
| (15) Benefit agent | — | | 0.01 | g | — | |
| (16) Benefit agent | — | | — | | 1.01 | g |
| Water | <5 | wt. % | <5 | wt. % | <5 | wt. % |
| % Acid converting agent | 22.7 | wt. % | 24.0 | wt. % | 15.8 | wt. % |
| % Benefit agent | 6.5 | wt. % | 0.5 | wt. % | 33.7 | wt. % |

Performance

| | | | |
|---|---|---|---|
| pH | 5.84 | 5.39 | 6.62 |

### Example 5: Through the Wash Fabric Treatment

Inventive examples demonstrate solid-dissolvable compositions composed of a mesh prepared from a blend of sodium fatty acid carboxylates, and then combined with acid converting agent and benefit agents to create a low pH aqueous treatment liquor when combined with water. These compositions are added to a washer drum in advance of the wash cycle, where the low pH aqueous treatment liquor reduces malodors.

In one embodiment (Examples EA-Examples ED), perfume capsules with different capsule architectures and/or pro-perfume (benefit agent) are incorporated into the substrate mesh. In a non-limiting case, the mesh is composed of a mixture of about 20 wt. % NaC8, 20% wt. % NaC10 and 60 wt. % NaC12. The substrate mesh are combined with acid converting agent and freshness agents, to create a solid-dissolvable composition. The solid-dissolvable composition is in the form of beads, about 20-30 mg. A number of solid-dissolvable composition are combined to create a metered single-measured dose which are placed in a cap, and then added to the wash cycle to enable low pH treatment liquor in the wash cycle. Such compositions enhance fabric freshness by reducing any malodor and enhancing freshness with freshness benefit agents.

In another embodiment, enhanced malodor compositions are created using compositions disclosure in Example EA-Example ED, replacing the freshness benefit agents with effective amounts of malodor benefit agents including polymer amine-based compounds.

In another embodiment, fabric feel compositions are created using compositions disclosure in Example EA-Example ED, replacing the freshness benefit agents with effective amounts of fabric feel benefit agents including quaternary ammonium compounds.

In another embodiment, fabric softening compositions are created using compositions disclosure in Example EA-Example ED, replacing the freshness benefit agents with effective amounts of fabric feel benefit agents including melting point fatty compounds and silicone compounds.

In another embodiment, fabric bleaching compositions are created using compositions disclosure in Example EA-Example ED, replacing the freshness benefit agents with effective amounts of fabric feel benefit agents including peroxyacid compounds.

In another embodiment, fabric cleaning compositions are created using compositions disclosure in Example EA-Example ED, replacing the freshness benefit agents with effective amounts of enzyme benefit agents including proteases compounds.

In another embodiment, fabric brightening compositions are created using compositions disclosure in Example EA-Example ED, replacing the freshness benefit agents with effective amounts of hueing agents benefit agents including small molecule dyes and polymeric dyes compounds.

In another embodiment, fabric microbial removal compositions are created using compositions disclosure in Example EA-Example ED, replacing the freshness benefit agents with effective amounts of antimicrobial, antibacterial, and antifungal compounds.

In another embodiment, perfume capsules with different capsule architectures and/or pro-perfume (benefit agent) are incorporated into the substrate mesh. The substrate mesh are combined with acid converting agent and freshness agents, to create a solid-dissolvable composition. The solid-dissolvable composition is in the form of thin pad, about 50 g. The pad serves as a metered single-measured dose which place in the wash cycle to enable low pH treatment liquor in the wash cycle. Such compositions enhance fabric freshness by reducing any malodor and enhancing freshness with freshness benefit agents.

TABLE 10

| | | | |
|---|---|---|---|
| Sample | Sample | Sample | Sample |
| EA | EB | EC | ED |
| Inventive | Inventive | Inventive | Inventive |

Substrate Mesh

| | | | | |
|---|---|---|---|---|
| Crystallizing | 75 wt. % | 75 wt. % | 65 wt. % | 55 wt. % |
| agent | | | | |
| Perfume capsule | 15 wt. % | 10 wt. % | 15 wt. % | 25 wt. % |
| from slurry | | | | |
| Pro-perfume | — | — | — | 30 wt. % |
| Acid converting | 10 wt. % | 15 wt. % | 20 wt. % | — |
| agent | | | | |
| Water | <5 wt. % | <5 wt. % | <5 wt. % | <5 wt. % |
| Bead Mass | 30 mg | 30 mg | 30 mg | 30 mg |
| % Acid | 10 wt. % | 10 wt. % | 15 wt. % | 25 wt. % |
| converting agent | | | | |
| % Benefit agent | 15 wt. % | 10 wt. % | 20 wt. % | 30 wt. % |

### Example 6: Through the Rinse Fabric Treatment

Solid-dissolvable composition that eliminates malodors from and imparts freshness (and other benefits) to laundry, whereas said composition is introduced into the drum of the washing machine through the fabric softener/conditioner compartment during the rinse cycle. The solid-dissolvable composition is pure white, and denotes a ‘natural’ look to the product. The crystallizing agents are “bio-based” and prepared from “renewable material” enhancing the real sustainability of the compositions.

In one embodiment (Example FA), the solid-dissolvable composition is composed of one substrate mesh of dimensions of about 6 cm×6 cm×1 cm, with perfume capsules dispersed throughout the mesh, and with the acid converting agent applied to one surface. The solid-dissolvable composition is added to the fabric softener/conditioner compartment at the start of the wash cycle. During the rinse cycle, water is passed over the solid-dissolvable composition completely dissolving the acid converting agent, and releasing the perfume capsules into the washer drum. The acid converting agent creates a low pH treatment liquor that eliminates malodors, and the perfume capsule then imparts freshness onto the fabric.

In another embodiment (Example FB), the solid-dissolvable composition is composed of two substrate meshes each with the dimensions of about 6 cm×6 cm×0.5 cm, with one type of perfume capsules dispersed throughout the first mesh and a second type of perfume capsule dispersed throughout the second mesh. The first substrate mesh comprises faster dissolving sodium fatty acid carboxylate, to release the first type of capsules earlier in the wash, while the second substrate mesh comprised slower dissolving sodium fatty acid carboxylate, to release the second type of capsules later in the wash. To assemble the solid-dissolvable composition, the acid converting agent is applied to the large surface of the first mesh substrate, and the second mesh substrate is affix on this mesh to create a “sandwich’ with the acid converting agent. The solid-dissolvable composition is added to the fabric softener/conditioner compartment at the start of the wash cycle. During the rinse cycle, water is passed over the solid-dissolvable composition completely dissolving the acid converting agent, and releasing the first and then the second set of perfume capsules perhaps five minutes later into the washer drum. The acid converting agent creates a low pH treatment liquor that eliminates malodors, and the perfume capsule then imparts blended freshness onto the fabric.

In another embodiment (Example FC), the solid-dissolvable composition is composed of one substrate mesh of dimensions of about 6 cm×6 cm×1 cm, with pro-perfume dispersed throughout the mesh, and with the acid converting agent applied to one surface. The solid-dissolvable composition is added to the fabric softener/conditioner compartment at the start of the wash cycle. During the rinse cycle, water is passed over the solid-dissolvable composition completely dissolving the acid converting agent, and releasing the perfume capsules into the washer drum. The acid converting agent creates a low pH treatment liquor that eliminates malodors, and the perfume capsule then imparts freshness onto the fabric.

In another embodiment (Example FD), the solid-dissolvable composition is composed of one substrate mesh of dimensions of a sphere about 2 cm in radius, with amino-silicone dispersed throughout the mesh, and with the acid converting agent applied to one surface. The solid-dissolvable composition is added to the fabric softener/conditioner compartment at the start of the wash cycle. During the rinse cycle, water is passed over the solid-dissolvable composition completely dissolving the acid converting agent, and releasing the silicone in drops into the washer drum for the proper size as not to stain fabrics (U.S. Pat. No. 10,301,575 B2) and without the potential for contact of the silicone that can also stain fabrics (U.S. Pat. No. 10,329,519 B2). The acid converting agent creates a low pH treatment liquor that eliminates malodors, and the silicone is a benefit agent that lowers friction and imparts fabric feel benefits.

In another embodiment (Example FE), the solid-dissolvable composition is composed of one substrate mesh of dimensions of a disk of about 2 cm in radius (ends) and about 1 cm in height, with otherwise unstable enzyme dispersed on one surface of the disk, and with the acid converting agent applied to the other surface of the disk (some enzymes are very useful but difficult to stabilize with a liquid formulation). The solid-dissolvable composition is added to the fabric softener/conditioner compartment at the start of the wash cycle. During the rinse cycle, water is passed over the solid-dissolvable composition completely dissolving the acid converting agent, and releasing the enzyme in drops into the washer drum where is can now safely hydrate and clean tough fabric stains. The acid converting agent creates a low pH treatment liquor that activates the enzymes, and the enzymes is a benefit agent that cleans difficult stains from fabrics.

TABLE 11

| | | | |
|---|---|---|---|
| Sample FA | Sample FB | Sample FC | Sample FD |
| Inventive | Inventive | Inventive | Inventive |

Substrate Mesh 1

| NaC8 | 45 g | 35 | g | 20 g | 75 g |
|---|---|---|---|---|---|
| NaC10 | 45 g | 35 | g | 20 g | — |
| NaC12 | — | — | | 60 g | 75 g |
| Perfume capsule from slurry (19) | 16 g | — | | — | — |
| Perfume capsule from slurry (20) | — | 30 | g | — | — |

| | | | | |
|---|---|---|---|---|
| Pro-perfume oil | — | — | 20 g | — |
| Aminosilicone | — | — | — | 15 g |
| Enzyme | — | — | — | — |

Substrate Mesh 2

| NaC8 | — | — | | — | — |
|---|---|---|---|---|---|
| NaC10 | — | 35 | g | — | — |
| NaC12 | — | 35 | g | — | — |
| Perfume capsule from slurry (19) | — | 30 | g | — | — |

| | | | | |
|---|---|---|---|---|
| Perfume capsule from slurry (20) | — | — | — | — |
| Pro-perfume oil | — | — | — | — |
| Aminosilicone | — | — | — | — |
| Enzyme | — | — | — | — |

SDC

| Mesh 1 | 18 g | 9 | g | 18 g | 34 g |
|---|---|---|---|---|---|
| Mesh 2 | — | 9 | g | — | — |
| (10) Acid Converting Agent | 18 g | — | | — | — |
| (11) Acid Converting Agent | — | 18 | g | — | — |

| (12) Acid Converting Agent | — | | — | 15 g | | 15 g | |
|---|---|---|---|---|---|---|---|
| (13) Acid Converting Agent | — | | — | — | | — | |
| % Acid converting agent | 50.0 | wt. % | 50.0 wt. % | 45.5 | wt. % | 30.6 | wt. % |
| % Benefit agent | 8.3 | wt. % | 15.0 wt. % | 9.1 | wt. % | 6.2 | wt. % |

TABLE 12

Sample FE
Inventive

Substrate Mesh 1

| NaC8 | 45 | g |
|---|---|---|
| NaC10 | 45 | g |
| NaC12 | — | |
| Perfume capsule from slurry (19) | — | |
| Perfume capsule from slurry (20) | — | |
| Pro-perfume oil | — | |
| Aminosilicone | — | |
| Enzyme | 2 | g |

Substrate Mesh 2

| | |
|---|---|
| NaC8 | — |
| NaC10 | — |
| NaC12 | — |
| Perfume capsule from slurry (19) | — |
| Perfume capsule from slurry (20) | — |
| Pro-perfume oil | — |
| Aminosilicone | — |
| Enzyme | — |

SDC

| Mesh 1 | 18 | g |
|---|---|---|
| Mesh 2 | — | |
| (10) Acid Converting Agent | 18 | g |
| (11) Acid Converting Agent | — | |
| (12) Acid Converting Agent | — | |
| (13) Acid Converting Agent | — | |
| % Acid converting agent | 50.0 | wt. % |
| % Benefit agent | 1.1 | wt. % |

### Example 7: Hair Treatment

Inventive examples demonstrate solid-dissolvable compositions composed of a mesh prepared from a blend of sodium fatty acid carboxylates, and then combined with acid converting agent and benefit agents to create a low pH aqueous treatment liquor when combined with water. These compositions are solid-form shampoos, which are added to the palm of the hand while in the shower, about 100 grams of water is added to the composition while in the hand, the consumer rubs hands together to create lather and then rubs the resulting composition over the hair and scalp. The natural PH balance with these compositions prevents hair damage and scalp irritation. The hair care benefit agent in the solid-dissolvable composition will enable other benefits to the hair and scalp. The sodium fatty acid carboxylate fiber mesh is resistant to humidity to ensure the composition maintains solid structure until mixing in the hand.

In one embodiment, the solid-dissolvable composition is composed of one substrate mesh of about 2.5 grams in the shape of an oval. The composition contains about 1.0 grams of substrate mesh comprising a blend of NaC8, NaC10 and NaC12, and 1.0 grams of acid converting agent, and about 0.5 grams of traditional ‘lathering’ surfactant.

In another embodiment, the solid-dissolvable composition design as a sulfate-free composition is composed of one substrate mesh of about 2.5 grams in the shape of a rectangle. The composition contains about 1.0 grams of substrate mesh comprising a blend of NaC8, NaC10 and NaC12, and 1.0 grams of acid converting agent, and about 0.5 grams of a blend of sulfate-free surfactant, such as lauramido propyl hydroxy sultaine.

In another embodiment, the solid-dissolvable composition design as a hair conditioning composition is composed of one substrate mesh of about 2.5 grams in the shape of a rectangle. The composition contains about 1.0 grams of substrate mesh comprising a blend of NaC8, NaC10 and NaC12, and 1.0 grams of acid converting agent, and about 0.5 grams of blend of a cationic polymers such as mono-long alkyl quaternized ammonium salt. In another embodiment, the benefit agent is a cationic polymer such as Polyquaternium-10. In another embodiment, the benefit agent is a co-polymer polymer such as PVP/VA copolymer. In another embodiment, the benefit agent is a blend of conditioning polymers.

In another embodiment, the solid-dissolvable composition design as anti-dandruff composition is composed of one substrate mesh of about 2.5 grams in the shape of a rectangle. The composition contains about 1.3 grams of substrate mesh comprising a blend of NaC8, NaC10 and NaC12, and 0.9 grams of acid converting agent, and about 0.3 grams of such as zinc pyrithione.

In another embodiment, the solid-dissolvable composition design as conditioning composition is composed of one substrate mesh of about 3.0 grams in the shape of a rectangle. The composition contains about 1.7 grams of substrate mesh comprising a blend of NaC8, NaC10 and NaC12, and 1.2 grams of acid converting agent, and about 0.2 grams of such as silicones such as dimethicone.

In another embodiment, the solid-dissolvable composition design as conditioning composition is composed of one substrate mesh of about 2.5 grams in the shape of a rectangle. The composition contains about 1.5 grams of substrate mesh comprising a blend of NaC8, NaC10 and NaC12, and 0.7 grams of acid converting agent, and about 0.3 grams of such as essential oils such as eucalyptus oil.

In another embodiment, the solid-dissolvable composition design as conditioning composition is composed of one substrate mesh of about 2.5 grams in the shape of a pad. The composition contains about 1.5 grams of substrate mesh comprising a blend of NaC8, NaC10 and NaC12, and 0.7 grams of acid converting agent, and about 0.3 grams of gel network.

In another embodiment, the solid-dissolvable composition design an absorbing composition is composed of one substrate mesh of about 3.5 grams in the shape of an oval. The composition contains about 2.0 grams of substrate mesh comprising a blend of NaC8, NaC10 and NaC12, and 1.2 grams of acid converting agent, and about 0.3 grams of such as sebum absorbing particle such as corn starch.

In another embodiment, the solid-dissolvable composition design to deliver freshness and conditioning to hair in two layers: one layer is composed of about 1.5 grams of substrate, acid converting agent and freshness agent (e.g., perfume capsules), one layer is composed of about 1.5 grams of substrate, acid converting agent and condition agent (e.g., Polyquaternium-10), where specific compositions as previously described. The two substrates are formed together, to create a single-measured composition.

### Example 8: Face/Skin Treatment

Inventive examples demonstrate solid-dissolvable compositions composed of a mesh prepared from a blend of sodium fatty acid carboxylates, and then combined with acid converting agent and benefit agents to create a low pH aqueous treatment liquor when combined with water. The skin pH enables cleaning and delivery of actives to enhance skin and skin health, while prevent any damage to the skin. These compositions are designed to wash and/or treat skin. The composition is added to the palm of the hand while at the sink, about 10 grams water is added to the composition while in the hand, the consumer rubs hands together to create a creamy lather as a low pH treatment liquor, and then is rubbed on the face and/or other the skin surfaces. The entire mixture is removed from the face and/or other skin surfaces by rinsing with water.

In one embodiment, the solid-dissolvable composition for healing and improving barrier function of skin is 5.0 grams and in the shape of an oval. The composition contains about 2.0 grams of substrate mesh comprising a blend of NaC8, NaC10 and NaC12, and 2.0 grams of lathering surfactant, and 1.0 grams of acid converting agent, and about 0.10 grams of actives from the Vitamin B3 family, including panthenol.

In another embodiment, the solid-dissolvable composition for helping protect the skin against water loss, which helps reduce dullness and promotes more radiant-looking skin which is combined about 5.0 grams in the shape of a pad. The composition contains about 2.0 grams of substrate mesh comprising a blend of NaC8, NaC10 and NaC12, and 2.0 grams of lathering surfactant, and 1.0 grams of acid converting agent, and about 0.10 grams of actives from the Vitamin C family, including sodium ascorbyl phosphate.

In another embodiment, the solid-dissolvable composition which renews your skin with powerful anti-aging ingredients skin composed of a substrate mesh, acid converting agent, and niacinamide (Vitamin B3), Vitamin C, collagen peptide, Vitamin E and alpha hydroxy acid (AHA), alone or in various combinations, thereof.

In another embodiment, the solid-dissolvable composition with powerful and effective ingredients to enhance the nourishing, toning, & regenerative properties of skin composed of about 2.0 grams of substrate mesh comprising a blend of NaC8, NaC10 and NaC12, and 1.0 grams of lathering surfactant, and 1.0 grams of acid converting agent, and about 4 wt. % natural extracts, including green tea extract.

### Example 9: Toilet Cleaner

Toilet cleaning products are formulated to facilitate cleaning the inside of bowls, and products require low pH treatment liquors to remove rust, mineral deposits, fecal matter, and other non-organic materials. The composition is added to the bowl and allowed to hydrate to release cleaning agents, and stains are removed optionally with a toilet brush.

Inventive examples demonstrate solid-dissolvable compositions of about 170 grams, composed of a mesh prepared from about 90 grams a blend of sodium fatty acid carboxylates, and about 10 grams of foaming surfactant, and about 60 grams acid converting agent, and about 5 grams effervescent agents and effervescent activator, and optionally about 5 grams of perfumes, soil release polymers, enzymes, bacterial spores, chelants, antimicrobial compounds, and/or bleaches.

What is claimed is:

1. A solid-dissolvable composition comprising:
crystallizing agent;
water;
acid converting agent;
wherein the crystallizing agent is the sodium salt of saturated fatty acids having from 8 to about 12 methylene groups; and
wherein the crystallizing agent is the form of fibers as determined by the FIBER TEST METHOD;
wherein the water is less than about 15 wt. % of the solid-dissolvable composition; and
wherein the acid converting agent is from about 10 wt. % to about 55 wt. % of the solid-dissolvable composition.

2. The solid-dissolvable composition of claim 1, wherein the crystallizing agent is from 50 wt. % to 90 wt. % of the composition.

3. The solid-dissolvable composition of claim 1, wherein the crystallizing agent comprises from 50 wt. % to 70 wt. % of a sodium salt of saturated fatty acids having 12 methylene groups; from 15 wt. % to 25 wt. % of a sodium salt of saturated fatty acids having 10 methylene groups; and from 15 wt. % to 25 wt. % of a sodium salt of saturated fatty acids having 8 methylene groups.

4. The solid-dissolvable composition of claim 1, wherein the composition has a low pH treatment liquor of pH less than 7, as determined by the PH TEST METHOD FOR WASH LIQUOR.

5. The solid-dissolvable composition of claim 1, wherein the composition has a low pH treatment liquor of pH less than 7 but more than 4, as determined by the PH TEST METHOD FOR WASH LIQUOR.

6. The solid-dissolvable composition of claim 1, wherein the acid converting agent is selected from citric acid, sodium citrate, succinic acid, succinate, salicylic acid, mandelic acid, oxalic acid, tartaric acid, malic acid, maleic acid, lactic acid, sodium malate, and mixtures thereof.

7. The solid-dissolvable composition of claim 1, further comprising a benefit agent.

8. The solid-dissolvable composition of claim 7, wherein the benefit agent is less than 35 wt. % of the solid-dissolvable composition.

9. The solid-dissolvable composition of claim 7, wherein the benefit agent is a hair treatment agent selected from zinc pyrithione, cationic quat polymers, cationic guars, cationic cellulosic, silicone oils, aminosilicone oils, and mixtures thereof.

10. The solid-dissolvable composition of claim 7, wherein the benefit agent is a fabric treatment agent selected from cationic quaternary surfactants, metathesized unsaturated polyol esters, hydrogenated oil, partially hydrogenated oils, polyglycerol esters (PGE), C60, LS660, cocoa butter, coconut oil, shea butter, natural extracts, co-polymer of acrylic acid and diallyldimethylammonium chloride (Polyquaternium-22), polyhydroxy stearic acid and Merquat 280.

11. The solid-dissolvable composition of claim 7, wherein the benefit agent is a skin care benefit agent.

12. The solid-dissolvable composition of claim 7, wherein the benefit agent is a toilet cleaning benefit agent.

13. The solid-dissolvable composition of claim 1, wherein the water is less than 5 wt. % of the composition.

14. The solid-dissolvable composition of claim 1, wherein the water is less than 10 wt. % of the composition.

15. The solid-dissolvable composition of claim 1, wherein the ratio of acid converting agent to crystallizing agent is from about 0.15 to about 1.2.

16. The solid-dissolvable composition of claim 1, wherein the acid converting agent is from about 20 wt. % to about 30 wt. % of the solid-dissolvable composition.

17. The solid-dissolvable composition of claim 1, wherein the crystallizing agent is from 70 wt. % to 80 wt. % of the composition.

18. A method for making the solid-dissolvable composition of claim 1, comprising the following steps:
preparing a substrate mesh;
drying the mesh;
adding acid converting agents; and
optionally adding one or more benefit agents.

19. A solid-dissolvable composition comprising:
crystallizing agent;
water;
acid converting agent;
wherein the crystallizing agent is the sodium salt of saturated fatty acids having from 8 to about 12 methylene groups; and
wherein the crystallizing agent is the form of fibers as determined by the FIBER TEST METHOD;
wherein the water is less than about 15 wt. % of the solid-dissolvable composition; and
wherein the composition has a low pH treatment liquor of pH less than 7, as determined by the PH TEST METHOD FOR WASH LIQUOR.

20. The solid-dissolvable composition of claim 19,
wherein the crystallizing agent is from 70 wt. % to 80 wt. % of the composition and is selected from sodium caprylic, sodium caprate, sodium laurate, and mixtures thereof;
wherein the acid converting agent is from about 20 wt. % to about 30 wt. % of the solid-dissolvable composition and is selected from citric acid, sodium citrate, succinic acid, succinate, salicylic acid, mandelic acid, oxalic acid, tartaric acid, malic acid, sodium malate, and mixtures thereof;
wherein the water is less than 5 wt. % of the composition; and
wherein the composition comprises a benefit agent.
